SEQUENCE LISTING (17 sequences)

SEQ ID NO: 1 - protein, 728 aa, synthetic construct
MGSSHHHHHHSSGLVPRGSHMVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKAAATENLYAVLQSGFRGSGSAMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGPRSGSLKGGAPTKASQEFMVSKGEELIKENMHMKLYMEGTVNNHHFKCTSEGEGKPYEGTQTMRIKVVEGGPLPFAFDILATSFMYGSKTFINHTQGIPDFFKQSFPEGFTWERSTTYEDGGVLTATQDTSLQDGCLIYNVKIRGVNFPSNGPVMQKKTLGWEASTEMLYPADGGLEGRDYMALKLVGGGHLICNAKTTYRSKKPAKNLKMPGVYYVDRRLERIKEADKETSVEQHEVAVARYCDLPSKLGHKGTDYKDHDGDYKDHDIDYKDDDDKDI

SEQ ID NO: 2 - DNA, 2188 bp, synthetic construct
  source 1..2188: mol_type = other DNA
atgggaagttcacatcatcatcatcatcactcatcaggactggtgccacgggggtctcacatggtctccaagggggaagaggacaacatggcctctctgcctgccacacacgagctgcacatcttcggcagcatcaatggcgtggactttgatatggtgggccagggcacaggcaacccaaatgacggctacgaggagctgaacctgaagagcaccaagggcgatctgcagttctccccttggatcctggtgccacacatcggctatggctttcaccagtatctgccctaccctgacggcatgtcccccttccaggccgccatggtggatggctctggctaccaggtgcacaggaccatgcagtttgaggacggcgcctccctgacagtgaattaccggtatacctacgagggctctcacatcaagggcgaggcccaggtgaagggcacaggcttcccagccgatggccccgtgatgacaaactctctgaccgccgccgactggtgccggagcaagaagacctaccctaatgataagacaatcatctccacctttaagtggtcttataccacaggcaacggcaagcggtacagaagcacagccagaaccacatatacctttgccaagccaatggccgccaactatctgaagaatcagcccatgtacgtgttcaggaagacagagctgaagcactccaagaccgagctgaatttcaaggagtggcagaaggcctttacagacgtgatgggcatggatgagctgtacaagggcggccgccaccgagaacctgtatgcagtgctccaaagcggatttcgcggctctggcagcgctatggtgttcacactggaggactttgtgggcgattggcggcagaccgccggctataatctggatcaggtgctggagcagggcggcgtgagctccctgttccagaacctgggcgtgagcgtgacccctatccagcggatcgtgctgtccggcgagaacggcctgaagatcgacatccacgtgatcatcccatacgagggcctgtctggcgatcagatgggccagatcgagaagatcttcaaggtggtgtaccccgtggacgatcaccacttcaaagtgatcctgcactatggcaccctggtcatcgacggcgtgacccccaatatgatcgattatttcggccggccttacgagggcatcgccgtgtttgacggcaagaagatcaccgtgacaggcaccctgtggaacggcaataagatcatcgacgagagactgatcaaccccgatggctccctgctgttcagggtgactattaacggagtgactggctggcggctgtgcgaaaggattctggctggaccgcggagcgggagcctgaaaggcggcgcgccgaccaaagcgagtcaggaattcatggtgtccaaaggcgaagaactgattaaagaaaatatgcatatgaaactgtatatggaaggcaccgtgaataatcatcattttaaatgcacctccgaaggcgaaggcaaaccgtatgaaggcacccagaccatgcgtattaaagtggtggaaggcggcccgctgccgtttgcgtttgatattctggcgacctcctttatgtatggctccaaaacctttattaatcatacccagggcattccggatttttttaaacagtcctttccggaaggctttacctgggaacgttccaccacctatgaagatggcggcgtgctgaccgcgacccaggatacctccctgcaggatggctgcctgatttataatgtgaaaattcgtggcgtgaattttccgtccaatggcccggtgatgcagaaaaaaaccctgggctgggaagcgtccaccgaaatgctgtatccggcggatggcggcctggaaggccgtgattatatggcgctgaaactggtgggcggcggccatctgatttgcaatgcgaaaaccacctatcgttccaaaaaaccggcgaaaaatctgaaaatgccgggcgtgtattatgtggatcgtcgtctggaacgtattaaagaagcggataaagaaacctccgtggaacagcatgaagtggcggtggcgcgttattgcgatctgccgtccaaactgggccataaaggtaccgactacaaagaccatgacggtgattataaagatcatgacatcgattacaaggatgacgatgacaaggatatctga

SEQ ID NO: 3 - protein, 730 aa, synthetic construct
MGSSHHHHHHSSGLVPRGSHMVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKAAATENLYAVLQSGFRGSGSAMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGPRSGSLKGGAPTKASQEFMVSKGEELIKENMHMKLYMEGTVNNHHFKCTSEGEGKPYEGTQTMRIKVVEGGPLPFAFDILATSFMYGSYTFINHTQGIPDFFKQSFPEGFTWERVTTYEDGGVLTATQDTSLQDGCLIYNVKIRGVNFTSNGPVMQKKTLGWEAGTEMLYPADGGLEGRSDDALKLVGGGHLICNLKSTYRSKKPAKNLKVPGVYYVDRRLERIKEADKETYVEQHEVAVARYCDLPSKLGHKLNGTDYKDHDGDYKDHDIDYKDDDDKDI

SEQ ID NO: 4 - DNA, 2193 bp, synthetic construct
  source 1..2193: mol_type = other DNA
atgggaagttcacatcatcatcatcatcactcatcaggactggtgccacgggggtctcacatggtctccaagggggaagaggacaacatggcctctctgcctgccacacacgagctgcacatcttcggcagcatcaatggcgtggactttgatatggtgggccagggcacaggcaacccaaatgacggctacgaggagctgaacctgaagagcaccaagggcgatctgcagttctccccttggatcctggtgccacacatcggctatggctttcaccagtatctgccctaccctgacggcatgtcccccttccaggccgccatggtggatggctctggctaccaggtgcacaggaccatgcagtttgaggacggcgcctccctgacagtgaattaccggtatacctacgagggctctcacatcaagggcgaggcccaggtgaagggcacaggcttcccagccgatggccccgtgatgacaaactctctgaccgccgccgactggtgccggagcaagaagacctaccctaatgataagacaatcatctccacctttaagtggtcttataccacaggcaacggcaagcggtacagaagcacagccagaaccacatatacctttgccaagccaatggccgccaactatctgaagaatcagcccatgtacgtgttcaggaagacagagctgaagcactccaagaccgagctgaatttcaaggagtggcagaaggcctttacagacgtgatgggcatggatgagctgtacaaggcggccgccaccgagaacctgtatgcagtgctccaaagcggatttcgcggctctggcagcgctatggtgttcacactggaggactttgtgggcgattggcggcagaccgccggctataatctggatcaggtgctggagcagggcggcgtgagctccctgttccagaacctgggcgtgagcgtgacccctatccagcggatcgtgctgtccggcgagaacggcctgaagatcgacatccacgtgatcatcccatacgagggcctgtctggcgatcagatgggccagatcgagaagatcttcaaggtggtgtaccccgtggacgatcaccacttcaaagtgatcctgcactatggcaccctggtcatcgacggcgtgacccccaatatgatcgattatttcggccggccttacgagggcatcgccgtgtttgacggcaagaagatcaccgtgacaggcaccctgtggaacggcaataagatcatcgacgagagactgatcaaccccgatggctccctgctgttcagggtgactattaacggagtgactggctggcggctgtgcgaaaggattctggctggaccgcggagcgggagcctgaaaggcggcgcgccgaccaaagcgagtcaggaattcatggtgtctaagggcgaagagctgattaaggagaacatgcacatgaagctgtacatggaaggcaccgtgaacaaccaccacttcaagtgcacatccgagggcgaaggcaagccctacgagggcacccagaccatgagaatcaaggtggtcgagggcggccctctacccttcgccttcgacatcttggctaccagcttcatgtacggcagctacaccttcatcaaccacacccagggcatccccgacttctttaagcagtccttccctgagggcttcacatgggagagagtcaccacatacgaagacgggggcgtgctgaccgctacccaggacaccagcctccaggacggttgcctcatctacaacgtcaagatcagaggggtgaacttcacatccaacggccctgtgatgcagaagaaaacactcggctgggaggccggcaccgagatgctgtaccccgctgacggcggcctggaaggcagatcggacgacgccctgaagctcgtgggcgggggccacctgatctgcaacttgaagagcacatacagatccaagaaacccgctaagaatctcaaggtgcccggcgtctactatgtggaccgaagactggaaagaatcaaggaggccgacaaagagacctacgtcgagcagcacgaggtggctgtggccagatactgcgacctccctagcaaactggggcacaagcttaatggtaccgactacaaagaccatgacggtgattataaagatcatgacatcgattacaaggatgacgatgacaaggatatctga

SEQ ID NO: 5 - protein, 728 aa, synthetic construct
MGSSHHHHHHSSGLVPRGSHMVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKAAATENLYAVLQSGFRGSGSAMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGPRSGSLKGGAPTKASQEFMVSKGEELIKENMRSKLYLEGSVNGHQFKCTHEGEGKPYEGKQTNRIKVVEGGPLPFAFDILATHFMYGSKVFIKYPADLPDYFKQSFPEGFTWERVMVFEDGGVLTATQDTSLQDGELIYNVKVRGVNFPANGPVMQKKTLGWEPSTETMYPADGGLEGRCDKALKLVGGGHLHVNFKTTYKSKKPVKMPGVHYVDRRLERIKEADNETYVEQYEHAVARYSNLGGGFTLEDFVGTDYKDHDGDYKDHDIDYKDDDDKDI

SEQ ID NO: 6 - DNA, 2187 bp, synthetic construct
  source 1..2187: mol_type = other DNA
atgggaagttcacatcatcatcatcatcactcatcaggactggtgccacgggggtctcacatggtctccaagggggaagaggacaacatggcctctctgcctgccacacacgagctgcacatcttcggcagcatcaatggcgtggactttgatatggtgggccagggcacaggcaacccaaatgacggctacgaggagctgaacctgaagagcaccaagggcgatctgcagttctccccttggatcctggtgccacacatcggctatggctttcaccagtatctgccctaccctgacggcatgtcccccttccaggccgccatggtggatggctctggctaccaggtgcacaggaccatgcagtttgaggacggcgcctccctgacagtgaattaccggtatacctacgagggctctcacatcaagggcgaggcccaggtgaagggcacaggcttcccagccgatggccccgtgatgacaaactctctgaccgccgccgactggtgccggagcaagaagacctaccctaatgataagacaatcatctccacctttaagtggtcttataccacaggcaacggcaagcggtacagaagcacagccagaaccacatatacctttgccaagccaatggccgccaactatctgaagaatcagcccatgtacgtgttcaggaagacagagctgaagcactccaagaccgagctgaatttcaaggagtggcagaaggcctttacagacgtgatgggcatggatgagctgtacaaggcggccgccaccgagaacctgtatgcagtgctccaaagcggatttcgcggctctggcagcgctatggtgttcacactggaggactttgtgggcgattggcggcagaccgccggctataatctggatcaggtgctggagcagggcggcgtgagctccctgttccagaacctgggcgtgagcgtgacccctatccagcggatcgtgctgtccggcgagaacggcctgaagatcgacatccacgtgatcatcccatacgagggcctgtctggcgatcagatgggccagatcgagaagatcttcaaggtggtgtaccccgtggacgatcaccacttcaaagtgatcctgcactatggcaccctggtcatcgacggcgtgacccccaatatgatcgattatttcggccggccttacgagggcatcgccgtgtttgacggcaagaagatcaccgtgacaggcaccctgtggaacggcaataagatcatcgacgagagactgatcaaccccgatggctccctgctgttcagggtgactattaacggagtgactggctggcggctgtgcgaaaggattctggctggaccgcggagcgggagcctgaaaggcggcgcgccgaccaaagcgagtcaggaattcatggtgagcaagggcgaggagctgatcaaggagaacatgagaagcaagctgtacctggaaggcagcgtgaacggccaccagttcaagtgcacccacgaaggggagggcaagccctacgagggcaagcagaccaacaggatcaaggtggtggagggaggccccctgccgttcgcattcgacatcctggccacccactttatgtacgggagcaaggtgttcatcaagtaccccgccgacctccccgattattttaagcagtccttccctgagggcttcacatgggagagagtcatggtgttcgaagacgggggcgtgctgaccgccacccaggacaccagcctccaggacggcgagctcatctacaacgtcaaggtcagaggggtgaacttcccagccaacggccccgtgatgcagaagaaaacactgggctgggagcccagcaccgagaccatgtaccccgctgacggcggcctggaaggcagatgcgacaaggccctgaagctcgtgggcgggggccacctgcacgtcaacttcaagaccacatacaagtccaagaaacccgtgaagatgcccggcgtccactacgtggaccgcagactggaaagaatcaaggaggccgacaacgagacctacgtcgagcagtacgagcacgctgtggccagatactccaacctgggcggaggcttcacactcgaagatttcgttggtaccgactacaaagaccatgacggtgattataaagatcatgacatcgattacaaggatgacgatgacaaggatatctga

SEQ ID NO: 7 - protein, 725 aa, synthetic construct
MGSSHHHHHHSSGLVPRGSHMVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKAAATENLYAVLQSGFRGSGSAMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGPRSGSLKGGAPTKASQEFMVSKGEAVIKEFMRFKVHMEGSMNGHEFEIEGEGEGRPYEGTQTAKLKVTKGGPLPFSWDILSPQFMYGSRAFTKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRKLDITSHNEDYTVVEQYERSEGRHSTGGMDELYKGTDYKDHDGDYKDHDIDYKDDDDKDI

SEQ ID NO: 8 - DNA, 2178 bp, synthetic construct
  source 1..2178: mol_type = other DNA
atgggaagttcacatcatcatcatcatcactcatcaggactggtgccacgggggtctcacatggtctccaagggggaagaggacaacatggcctctctgcctgccacacacgagctgcacatcttcggcagcatcaatggcgtggactttgatatggtgggccagggcacaggcaacccaaatgacggctacgaggagctgaacctgaagagcaccaagggcgatctgcagttctccccttggatcctggtgccacacatcggctatggctttcaccagtatctgccctaccctgacggcatgtcccccttccaggccgccatggtggatggctctggctaccaggtgcacaggaccatgcagtttgaggacggcgcctccctgacagtgaattaccggtatacctacgagggctctcacatcaagggcgaggcccaggtgaagggcacaggcttcccagccgatggccccgtgatgacaaactctctgaccgccgccgactggtgccggagcaagaagacctaccctaatgataagacaatcatctccacctttaagtggtcttataccacaggcaacggcaagcggtacagaagcacagccagaaccacatatacctttgccaagccaatggccgccaactatctgaagaatcagcccatgtacgtgttcaggaagacagagctgaagcactccaagaccgagctgaatttcaaggagtggcagaaggcctttacagacgtgatgggcatggatgagctgtacaaggcggccgccaccgagaacctgtatgcagtgctccaaagcggatttcgcggctctggcagcgctatggtgttcacactggaggactttgtgggcgattggcggcagaccgccggctataatctggatcaggtgctggagcagggcggcgtgagctccctgttccagaacctgggcgtgagcgtgacccctatccagcggatcgtgctgtccggcgagaacggcctgaagatcgacatccacgtgatcatcccatacgagggcctgtctggcgatcagatgggccagatcgagaagatcttcaaggtggtgtaccccgtggacgatcaccacttcaaagtgatcctgcactatggcaccctggtcatcgacggcgtgacccccaatatgatcgattatttcggccggccttacgagggcatcgccgtgtttgacggcaagaagatcaccgtgacaggcaccctgtggaacggcaataagatcatcgacgagagactgatcaaccccgatggctccctgctgttcagggtgactattaacggagtgactggctggcggctgtgcgaaaggattctggctggaccgcggagcgggagcctgaaaggcggcgcgccgaccaaagcgagtcaggaattcatggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcaccaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagggtaccgactacaaagaccatgacggtgattataaagatcatgacatcgattacaaggatgacgatgacaaggatatctga

SEQ ID NO: 9 - protein, 8 aa, synthetic construct
AVLQSGFR

SEQ ID NO: 10 - DNA, 24 bp, synthetic construct
  source 1..24: mol_type = other DNA
gcagtgctccaaagcggatttcgc

SEQ ID NO: 11 - protein, 8 aa, synthetic construct
LKGGAPTK

SEQ ID NO: 12 - DNA, 24 bp, synthetic construct
  source 1..24: mol_type = other DNA
ctgaaaggcggcgcgccgaccaaa

SEQ ID NO: 13 - protein, 8 aa, synthetic construct
AVLASGFR

SEQ ID NO: 14 - DNA, 24 bp, synthetic construct
  source 1..24: mol_type = other DNA
gcagtgctcgccagcggatttcgc

SEQ ID NO: 15 - protein, 8 aa, synthetic construct
LKGIAPTK

SEQ ID NO: 16 - DNA, 24 bp, synthetic construct
  source 1..24: mol_type = other DNA
ctgaaaggcattgcgccgaccaaa

SEQ ID NO: 17 - protein, 8 aa, synthetic construct
DYKDDDDK

(19) United States
(12) Patent Application Publication
(10) Pub. No.: US 2025/0035622 A1
(43) Pub. Date: Jan. 30, 2025

(54) BRET-BASED, DUAL PROTEASE BIOSENSOR

(71) Applicant: Qatar Foundation for Education, Science and Community Development, Doha (QA)

(72) Inventors: Kabir H. Biswas, Doha (QA); Asma Fatima, Doha (QA); Anupriya M. Geethakumari, Doha (QA)

(21) Appl. No.: 18/763,140

(22) Filed: Jul. 3, 2024

Related U.S. Application Data
(60) Provisional application No. 63/525,307, filed on Jul. 6, 2023.

(51) Int. Cl. G01N 33/543; G01N 33/569

(52) U.S. Cl. CPC G01N 33/54373; G01N 33/56983; G01N 2333/165; G01N 2333/9513

(57) ABSTRACT

A biosensor for detecting proteolytic cleavage activity of two proteases, coronavirus main protease (M^{pro}) and papain-like protease (PL^{pro}) is provided. The biosensor utilizes Bioluminescence Resonance Energy Transfer (BRET) between a donor and two acceptor moieties.

### CROSS REFERENCE TO RELATED APPLICATIONS

This application claims the benefit of U.S. Provisional Application No. 63/525,307 filed Jul. 6, 2023, which is incorporated herein by reference in its entirety.

### SEQUENCE LISTING

This application contains a sequence listing having the filename 5600234-01187_Sequence_Listing.xml, which is 26.8 KB in size, and was created on Jun. 24, 2024. The entire content of this sequence listing is incorporated herein by reference.

### FIELD

The present disclosure relates to a biosensor which utilizes Bioluminescence Resonance Energy Transfer (BRET) and can simultaneously detect two proteins (e.g., two proteases).

### BACKGROUND

Bioluminescence Resonance Energy Transfer (BRET) is a phenomenon of proximity-dependent non-radiative, Forster resonance energy transfer (FRET) between a bioluminescent luciferase donor protein and a fluorescent acceptor protein. Besides physical proximity, the efficiency of BRET depends on the spectral overlap of the donor emission spectra and acceptor excitation spectra and the relative orientation of the donor and the acceptor moieties. It is a naturally occurring phenomenon and can be observed, for instance, between the jellyfish genus Aequorea, photoprotein aequorin, which emits blue light, and the green fluorescent protein (GFP) leading to the production of green light. BRET was initially utilized for studying interactions between the cyanobacterial circadian proteins KaiA and KaiB leading to the observation of KaiB homodimerization. Following this initial report, BRET has been utilized in developing a large variety of biological and biomedical applications. Some key examples of assays where BRET has been utilized successfully include monitoring protein-protein interactions such as the formation of multiprotein complexes by G-protein coupled receptors (GPCRs), detecting protein conformational changes, engineering biosensors for detecting biomolecules such as cAMP and cGMP, ATP, maltose or ions such as Ca²⁺ and Zn²⁺, detecting receptor-ligand binding, opto-genetics using the light-oxygen-voltage-sensing (LOV) domain protein, or monitoring the activity of proteolytic enzymes.

These successful applications of BRET have been largely due to significant advances in the engineering of brighter and smaller luciferases such as mutants of the Renilla luciferase, NanoLuc (NLuc), and more recently, artificial luciferases such as picALuc and its mutants, luciferase and their substrates with altered emission spectra, spectrally shifted and/or brighter fluorescent acceptor proteins such as GFP2 or mNeonGreen (mNG). In addition to these, recently a combination of BRET and fluorescence resonance energy transfer has been utilized to develop a number of applications including biosensors such as those for extracellular regulating kinase (ERK), cAMP, Zn²⁺, development of optical encryption keys using dendrimeric DNA-based nanoscaffold, for visualizing tissue inflammation and others. While these have undoubtedly increased the applications of BRET, they are limited to the utilization of a single acceptor fluorescent protein and therefore, BRET assays have been limited to reporting only one type of molecular event.

Thus, there is a need to expand this technology so that more than one type of molecular event is reported.

### SUMMARY

This disclosure provides an extension of bioluminescence resonance energy transfer (BRET) from the utilization of a single acceptor to two acceptor moieties in a single biosensor construct, thereby allowing for simultaneous monitoring of the activity of two proteins in a living cell. Provided herein is a BRET sensor comprising: 1) a bioluminescent protein, or mutants thereof; 2) a first BRET acceptor comprising a monomeric mNeonGreen (mNG) fluorescent protein; and 3) a second BRET acceptor comprising a red fluorescent protein (RFP); wherein the sensor detects the activity of two proteins as described herein. In some embodiments, the activity is proteolytic cleavage, and the sensor detects the activity of two proteases.

Provided is a dual protease sensor for detecting the coronavirus proteases comprising an amino acid construct of SEQ ID NO: 1, SEQ ID NO: 3, SEQ ID NO: 5, or SEQ ID NO: 7.

Further provided is a plasmid comprising a nucleotide construct of SEQ ID NO: 2, SEQ ID NO: 4, SEQ ID NO: 6, or SEQ ID NO: 8.

Also provided is a cell transfected with a plasmid comprising a nucleotide sequence of SEQ ID NO: 2, SEQ ID NO: 4, SEQ ID NO: 6, or SEQ ID NO: 8.

### BRIEF DESCRIPTION OF THE DRAWINGS

The following figures are included to illustrate certain aspects of the present disclosure and should not be viewed as exclusive embodiments. The subject matter disclosed is capable of considerable modifications, alterations, combinations, and equivalents in form and function, as will occur to one having ordinary skill in the art and having the benefit of this disclosure.

FIG. 1. depicts a BRET-based, dual protease biosensor (DuProSense). Shown in FIG. 1 is a schematic representation of the design and activity of genetically encoded, BRET-based SARS-CoV-2 dual proteases activity biosensor expressed in live cells. Close positioning (e.g., approximately 10 nm) of the mNG and RFP with NLuc results in a significant resonance energy transfer in the absence of the SARS-CoV-2 M^{pro} and PL^{pro} activity. The activity of the SARS-CoV-2 M^{pro} protease results in the cleavage of the biosensor resulting in a decrease in the resonance energy transfer between NLuc and mNG, leading to a reduction of the green fluorescence of the biosensor. The SARS-CoV-2 PL^{pro} activity results in the cleavage of the biosensor resulting in the reduction of BRET between NLuc and RFP, leading to a decrease in the red fluorescence of the biosensor.

FIG. 2A shows a schematic of the dual protease biosensor constructs containing the indicated red fluorescent protein (RFP) (mBeRFP, LLS-mKate2, CyOFP1 or mScarlet) in addition to mNG and NLuc.

FIG. 2B shows graphs of NLuc bioluminescence spectra along with the emission and excitation of mNG and indicated RFP (mBeRFP, LLS-mKate2, CyOFP1 or mScarlet).

FIG. 2C shows graphs of fluorescence spectra for the constructs shown in FIG. 2A. Fluorescence spectra was acquired at an excitation wavelength of 440 nm.

FIG. 2D shows graphs of bioluminescence emission spectra of the dual protease biosensor constructs containing mBeRFP, LLS-mKate2, CyOFP1, and mScarlet RFPs.

FIG. 2E shows green channel BRET data for the constructs of FIG. 2A. Data shown are mean±s.d. obtained from three independent experiments, with each experiment performed in triplicates.

FIG. 2F shows red channel BRET data for the constructs of FIG. 2A. Data shown are mean±s.d. obtained from three independent experiments, with each experiment performed in triplicates.

FIG. 2G shows a table showing p-values obtained for M^{pro} and PL^{pro} biosensor BRET obtained from a one-way ANOVA with Tukey's multiple comparison test.

FIG. 3A shows M^{pro} biosensor BRET (bar graphs, left panel), and change in M^{pro} biosensor BRET (dotted graphs, right panel) for dual protease biosensors comprising mBeRFP in the absence of any protease or in the presence of either M^{pro} or PL^{pro} or both in living HEK293T cells.

FIG. 3B shows PL^{pro} biosensor BRET (bar graphs, left panel), and change in PL^{pro} biosensor BRET (dotted graphs, right panel) for dual protease inhibitors comprising mBeRFP in the absence of any protease or in the presence of either M^{pro} or PL^{pro} or both in living HEK293T cells.

FIG. 3C shows M^{pro} biosensor BRET (bar graphs, left panel), and change in M^{pro} biosensor BRET (dotted graphs, right panel) for dual protease biosensors comprising LSS-mKate2 in the absence of any protease or in the presence of either M^{pro} or PL^{pro} or both in living HEK293T cells.

FIG. 3D shows PL^{pro} biosensor BRET (bar graphs, left panel), and change in PL^{pro} biosensor BRET (dotted graphs, right panel) for dual protease inhibitors comprising LSS-mKate2 in the absence of any protease or in the presence of either M^{pro} or PL^{pro} or both in living HEK293T cells.

FIG. 3E shows M^{pro} biosensor BRET (bar graphs, left panel), and change in M^{pro} biosensor BRET (dotted graphs, right panel) for dual protease biosensors comprising CyOFP1 in the absence of any protease or in the presence of either M^{pro} or PL^{pro} or both in living HEK293T cells.

FIG. 3F shows PL^{pro} biosensor BRET (bar graphs, left panel), and change in PL^{pro} biosensor BRET (dotted graphs, right panel) for dual protease inhibitors comprising CyOFP1 in the absence of any protease or in the presence of either M^{pro} or PL^{pro} or both in living HEK293T cells.

FIG. 3G shows M^{pro} biosensor BRET (bar graphs, left panel), and change in M^{pro} biosensor BRET (dotted graphs, right panel) for dual protease biosensors comprising mScarlet in the absence of any protease or in the presence of either M^{pro} or PL^{pro} or both in living HEK293T cells.

FIG. 3H shows PL^{pro} biosensor BRET (bar graphs, left panel), and change in PL^{pro} biosensor BRET (dotted graphs, right panel) for dual protease inhibitors comprising mScarlet in the absence of any protease or in the presence of either M^{pro} or PL^{pro} or both in living HEK293T cells. There is a specific and high decrease in both the M^{pro} (green channel) and PL^{pro} (red channel) BRET of the mScarlet containing dual protease biosensor (bottom panel). Data shown in FIG. 3A-H are mean±s.d. obtained from three independent experiments, with each experiment performed in triplicates. Accordingly, mScarlet-based DuProSense enables robust SARS-CoV-2 M^{pro} and PL^{pro} activity detection in live cells.

FIG. 4A depicts anti-FLAG-tag western blot images showing proteolytic cleavage of mBeRFP containing DuProSense constructs in the absence of M^{pro} and PL^{pro} (first lane), in the presence of M^{pro} alone (second lane), PL^{pro} alone (third lane) and both M^{pro} as well as PL^{pro} (fourth lane). Predicted molecular weights of the DuProSense constructs and proteolytic cleavage products are shown on the right side.

FIG. 4B depicts anti-FLAG-tag western blot images showing proteolytic cleavage of LSS-mKate2 containing DuProSense constructs in the absence of M^{pro} and PL^{pro} (first lane), in the presence of M^{pro} alone (second lane), PL^{pro} alone (third lane) and both M^{pro} as well as PL^{pro} (fourth lane). Predicted molecular weights of the DuProSense constructs and proteolytic cleavage products are shown on the right side.

FIG. 4C depicts anti-FLAG-tag western blot images showing proteolytic cleavage of CyOFP1 containing DuProSense constructs in the absence of M^{pro} and PL^{pro} (first lane), in the presence of M^{pro} alone (second lane), PL^{pro} alone (third lane) and both M^{pro} as well as PL^{pro} (fourth lane). Predicted molecular weights of the DuProSense constructs and proteolytic cleavage products are shown on the right side.

FIG. 4D depicts anti-FLAG-tag western blot images showing proteolytic cleavage of mScarlet containing DuProSense constructs in the absence of M^{pro} and PL^{pro} (first lane), in the presence of M^{pro} alone (second lane), PL^{pro} alone (third lane) and both M^{pro} as well as PL^{pro} (fourth lane). Predicted molecular weights of the DuProSense constructs and proteolytic cleavage products are shown on the right side. Accordingly, FIGS. 4A-D reveal cleavage of the constructs in the presence of proteases.

FIG. 5A shows the cleavage of the SARS-CoV-2 M^{pro} cleavage site and the DuProSense BRET response of the mScarlet-containing dual protease biosensor containing both M^{pro} and PL^{pro} cleavage sites. M^{pro} biosensor BRET (bar graphs, left panel) and change in M^{pro} biosensor BRET (dotted graphs, right panel) in the absence of any protease or in the presence of either M^{pro} or PL^{pro} or both in living HEK293T cells are shown.

FIG. 5B shows the cleavage of the SARS-CoV-2 PL^{pro} cleavage site and the DuProSense BRET response of the mScarlet-containing dual protease biosensor containing both M^{pro} and PL^{pro} cleavage sites. PL^{pro} biosensor BRET (bar graphs, left panel) and change in PL^{pro} biosensor BRET (dotted graphs, right panel) in the absence of any protease or in the presence of either M^{pro} or PL^{pro} or both in living HEK293T cells are shown.

FIG. 5C shows that mutation in the SARS-CoV-2 M^{pro} cleavage site abrogates DuProSense M^{pro} biosensor BRET response of the mScarlet-containing dual protease biosensor containing only PL^{pro} cleavage site (M^{pro} mutant). M^{pro} biosensor BRET (bar graphs, left panel) and change in M^{pro} biosensor BRET (dotted graphs, right panel) in the absence of any protease or in the presence of either M^{pro} or PL^{pro} or both in living HEK293T cells are shown.

FIG. 5D shows that mutation in the SARS-CoV-2 M^{pro} cleavage site does not affect the DuProSense PL^{pro} biosensor BRET response of the mScarlet-containing dual protease biosensor containing only PL^{pro} cleavage site (M^{pro} mutant). PL^{pro} biosensor BRET (bar graphs, left panel) and change in PL^{pro} biosensor BRET (dotted graphs, right panel) in the absence of any protease or in the presence of either M^{pro} or PL^{pro} or both in living HEK293T cells are shown.

FIG. 5E shows that mutation in the SARS-CoV-2 PL^{pro} cleavage site does not affect the DuProSense M^{pro} biosensor BRET response of the mScarlet-containing dual protease biosensor containing only M^{pro} cleavage site (PL^{pro} mutant). M^{pro} biosensor BRET (bar graphs, left panel) and change in M^{pro} biosensor BRET (dotted graphs, right panel) in the absence of any protease or in the presence of either M^{pro} or PL^{pro} or both in living HEK293T cells are shown.

FIG. 5F shows that mutation in the SARS-CoV-2 PL^{pro} cleavage site abrogates DuProSense PL^{pro} biosensor BRET response of the mScarlet-containing dual protease biosensor containing only M^{pro} cleavage site (PL^{pro} mutant). PL^{pro} biosensor BRET (bar graphs, left panel) and change in PL^{pro} biosensor BRET (dotted graphs, right panel) in the absence of any protease or in the presence of either M^{pro} or PL^{pro} or both in living HEK293T cells are shown.

FIG. 5G shows that mutations in SARS-CoV-2 M^{pro} and PL^{pro} cleavage sites abrogate DuProSense BRET response of the mScarlet-containing dual protease biosensor containing neither M^{pro} nor PL^{pro} cleavage sites (both M^{pro}+PL^{pro} mutant). M^{pro} biosensor BRET (bar graphs, left panel) and change in M^{pro} biosensor BRET (dotted graphs, right panel) in the absence of any protease or in the presence of either M^{pro} or PL^{pro} or both in living HEK293T cells are shown.

FIG. 5H shows that mutations in SARS-CoV-2 M^{pro} and PL^{pro} cleavage sites abrogate DuProSense BRET response of the mScarlet-containing dual protease biosensor containing only neither M^{pro} nor PL^{pro} cleavage sites (both M^{pro}+PL^{pro} mutant). PL^{pro} biosensor BRET (bar graphs, left panel) and change in PL^{pro} biosensor BRET (dotted graphs, right panel) in the absence of any protease or in the presence of either M^{pro} or PL^{pro} or both in HEK293T cells are shown. Data shown in FIG. 5A-H are mean±s.d. obtained from three independent experiments, with each experiment performed in triplicates.

FIG. 6A depicts graphs showing M^{pro} biosensor BRET in the presence of M^{pro} (circles) in cells expressing the mScarlet-containing dual protease biosensor and treated with the indicated concentrations of GC376 (M^{pro} inhibitor) simultaneously with transfection. For FIGS. 6A-F, BRET ratio in the absence of any protease is shown in blank circles while those in the presence of GRL-0617 are shown in blank or hashed squares, and those in the presence of GC376 are shown in dotted or hashed circles.

FIG. 6B provides graphs showing M^{pro} biosensor BRET in the presence of PL^{pro} (squares) in cells expressing the mScarlet containing dual protease biosensor and treated with the indicated concentrations of GRL-0617 (PL^{pro} inhibitor) simultaneously with transfection.

FIG. 6C provides graphs showing PL^{pro} biosensor BRET in the presence of M^{pro}(circles) in cells expressing the mScarlet containing dual protease biosensor and treated with the indicated concentrations of GC376 (M^{pro} inhibitor) simultaneously with transfection.

FIG. 6D provides graphs showing PL^{pro} biosensor BRET in the presence of PL^{pro} (circles) in cells expressing the mScarlet containing dual protease biosensor and treated with the indicated concentrations of GRL-0617 (PL^{pro} inhibitor) simultaneously with transfection.

FIG. 6E shows percentage M^{pro} activity in cells treated with the indicated concentrations of GC376.

FIG. 6F shows percentage PL^{pro} activity in cells treated with the indicated concentrations of GRL-0617. Inset in each graph shows log(EC₅₀) values for the inhibitor. Data in FIG. 6A-F are mean±s.d. from three independent experiments, with each experiment performed in triplicates.

### DETAILED DESCRIPTION

There are several advantages of Bioluminescence Resonance Energy Transfer (BRET) over the widely used, competing technology, Fluorescence Resonance Energy Transfer (FRET), wherein resonance energy transfer occurs between two fluorescent proteins acting as an energy donor and an energy acceptor. Some of these advantages include no requirement for an external light source for donor fluorescent protein excitation and low non-specific emission in the acceptor channel that is typically observed in in FRET experiments, either due to bleed through from optical filters or direct excitation of the acceptor fluorescent protein.

Bioluminescence Resonance Energy Transfer between a donor and an acceptor moiety has been successfully utilized in designing a wide range of molecular assays including monitoring protein-protein interaction, protein conformational changes, and proteolytic cleavage. However, these have been limited to the detection of a single molecular event involving the use of a single acceptor moiety.

One characteristic of coronaviruses is the presence of two proteases termed main protease (M^{pro}) and papain-like protease (PL^{pro}). Coronaviruses are single-stranded RNA viruses that cause respiratory tract diseases in mammals and birds. Coronaviruses are divided into four genera, alphacoronavirues, betacoronaviruses, gammacoronaviruses, and deltacoronaviruses. Non-limiting examples of coronaviruses include porcine epidemic diarrhea virus, Middle East respiratory syndrome-related coronavirus (MERS), severe acute respiratory syndrome-related coronaviruses (SARS-CoV-1 and SARS-CoV-2)

As emerging coronaviruses are increasingly causing serious disease in birds, humans, and other mammals, new treatments for these viruses are needed. The M^{pro} and PL^{pro} proteases are important as therapeutic targets for treating coronavirus. Thus, the presently disclosed biosensor was developed to allow the determination of interactions with two proteases in a single assay.

Here, we report the extension of BRET from the utilization of a single acceptor to two acceptor moieties in a single biosensor construct. Specifically, we designed DuProSense, a BRET-based, dual protease activity biosensor by utilizing two acceptor fluorescent proteins with distinct spectral characteristics. For this, we utilized an N-terminal fusion of mNeonGreen (mNG) as the acceptor in the green channel and a C-terminal fusion of a red fluorescent protein (RFPs) as the acceptor in the red channel while nanoLuc (NLuc) served as the donor for both channels. We included the SARS-CoV-2 M^{pro} and PL^{pro} cleavage sites sandwiched between mNG and NLuc and NLuc and RFP, respectively. Expression of the biosensor along with either M^{pro} or PL^{pro} specifically resulted in a decrease in the green and red channel BRET (after considering FRET between the acceptor fluorescent proteins), respectively, while expression of both M^{pro} and PL^{pro} resulted in a decrease in BRET in both channels. Mutations in the M^{pro} and PL^{pro} cleavage sites individually abrogated BRET changes in the green and red channels, respectively, while mutations in both M^{pro} and PL^{pro} cleavage sites abrogated BRET changes in both channels. Finally, we tested DuProSense to detect concentration-dependent inhibition of both M^{pro} and PL^{pro} using their known pharmacological inhibitors GC376 and GRL-0617, respectively. The BRET-based dual protease biosensor described herein, DuProSense, will be applicable in simultaneously monitoring the proteolytic cleavage activity of other pairs of proteases as well as screening pharmacological inhibitors of the proteases.

We increased the applicability of BRET through the utilization of two spectrally distinct fluorescent acceptor proteins for simultaneous monitoring of two types of molecular events through determining resonance energy transfer to the acceptor fluorescent proteins in a single biosensor construct. For this, we utilize a BRET donor-acceptor pair consisting of NLuc luciferase and mNG green fluorescent protein for monitoring BRET in the green channel and a red fluorescent protein as the second acceptor for monitoring BRET in the red channel. Additionally, we tested and validated the biosensing strategy by designing a dual protease biosensor for the simultaneous monitoring of SARS-CoV-2 main protease (M^{pro} or 3CL^{pro} or non-structural protein, NSP5) and papain-like protease (PL^{pro} or NSP3) proteolytic activity. SARS-CoV-2 infection cycle requires the proteolytic cleavage of the polyproteins pp1a and pp1ab through the activity of the two viral cysteine proteases, M^{pro} and PL^{pro}. Specifically, the SARS-CoV-2 genome-encoded polyproteins pp1a and pp1ab consisting of a total of 16 proteins (NSP1 to NSP16 with pp1a encoding for proteins NSP1 to NSP 11 while pp1ab encoding for proteins NSP1 to NSP16). In these, M^{pro} cleaves the polyproteins at a total of 11 sites (present between NSP4 to NSP11 in pp1a and NSP4 to NSP16 in pp1ab) while PL^{pro} cleaves the polyproteins at a total of 3 sites (present between NSP1 to NSP4 in both pp1a and pp1ab). In addition to the cognate cleavage sites present in the SARS-CoV-2 polyproteins, these proteases are also known to proteolytically cleave some host proteins. For instance, PL^{pro} is known to cleave the interferon-stimulated gene 15 product (ISG15) resulting in its deubiquitylation and leading to dysregulation of the host innate immune response by the protein.

Coronavirus proteases show high specificity with regards to their substrate peptide sequence and catalytic activity distinct from those of host proteases. For these reasons, the proteases can be suitable targets for the development of pharmacological agents towards coronaviruses, including SARS-CoV-2. For example, nirmatrelvir, an orally active M^{pro} inhibitor developed by Pfizer has been approved by the United States Food and Drug Administration (FDA) for the treatment of SARS-CoV-2.

There is a need for the development of other pharmacological inhibitors that target coronaviruses, including SARS-CoV-2. Agents that can simultaneously target both proteases (bispecific inhibitors) are a viable alternative for combating coronavirus infections. Bispecific pharmacological inhibitors of coronaviruses proteases will be of particular importance given the emergence of SARS-CoV-2 variants with increased infection potential, disease severity, and resistance to antibody-mediated neutralization developed through the currently administered vaccines.

Additionally, there is also a need for the functional characterization of mutations in the M^{pro} and PL^{pro} proteases that are observed in the coronaviruses, including SARS-CoV-2. While several assays are available to monitor the activity of these proteases, these are often marred by factors such as high cost, low specificity, and sensitivity. Therefore, the engineering of a BRET-based M^{pro} and PL^{pro} dual protease biosensor (DuProSense) is of interest because of its ability to enable simultaneous monitoring of the activity of multiple proteases and, therefore, the activity of pharmacological agents against the proteases or mutations in the proteases.

The development of dual protease sensors requires monitoring specific resonance energy transfer and spectral de-mixing of mNG and RFP fluorescence signals from the same samples to monitor the activity of both proteases simultaneously. In addition to detection to two proteases simultaneously, it will be appreciated that any two proteins may be detected simultaneously by constructing dual sensors in the manner provided herein. Accordingly, the methods described herein are not limited to proteases and/or SARS-CoV-2 detection, rather, the methods described herein can also be used for simultaneous monitoring of other pairs of proteins and/or diseases, and such embodiments are also contemplated within the scope of embodiments provided herein.

Described herein is a BRET-based dual protease biosensor comprising cognate cleavage sites of M^{pro} and PL^{pro} proteases between mNG and NLuc (green channel) and NLuc and an RFP (red channel), respectively. Based on the results obtained from fluorescence, bioluminescence, and BRET measurements with biosensors constructed using RFPs, including long Stoke-shifted RFPs (mBeRFP, LSS-mKate2, CyOFP1, and mScarlet). In some embodiments, the RFP is mScarlet. The specificity and sensitivity of the mNG and mScarlet-based DuProSense were determined through mutagenesis and known pharmacological inhibitors.

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

“CyOFP1” refers to a basic (constitutively fluorescent) long stokes shift fluorescent protein published in 2016, derived from Entacmaea quadricolor (Nature Biotechnology, 2016, 34(7):760-767). “mScarlet” refers to a basic (constitutively fluorescent) red fluorescent protein derived from synthetic construct (Nature Methods, 2016, 14(1):53-56). “LSS-mKate2” refers to a basic (constitutively fluorescent) long stokes shift fluorescent protein derived from Entacmaea quadricolor (Proceedings of the National Academy of Sciences, 2010, 107(12):5369-5374). “mBeRFP” refers to a basic (constitutively fluorescent) long stokes shift fluorescent protein derived from Entacmaea quadricolor (PLoS ONE, 2013, 8(6):e64849).

“NanoLuc (NLuc)” refers to a luciferase of about 19 kDa, or mutants thereof, developed from a deep-sea shrimp (ACS Chemical Biology, 2012, 7:848-1857; Bioconjugate Chemistry, 2016, 27:1175-1187).

“picALuc” refers to a small artificial luciferase of about 13 kDa, or mutants thereof (ACS Chemical Biology, 2022, 17:864-872; Bioconjug Chem 2013, 24:2067-2075; bioRxiv, 2023, doi:https://doi.org/10.1101/2023.02.14.528398). JP2014-100137 (PTL 1) and WO 2017/057752 (PTL 2) disclose an artificial luciferase (“ALuc”) engineered by selecting frequent amino acids from the amino acid sequence of a copepod-derived luciferase, which are incorporated herein by reference for all they disclose regarding ALuc.

“Sensor” is used interchangeably with biosensor.

“Red channel” refers to the measurement of fluorescence from a red fluorescent protein (RFP) acceptor in the sensor.

“Green channel” refers to the measurement of fluorescence from a green fluorescent protein (mNG protein) acceptor in the sensor.

GC376 refers to a compound of structure:

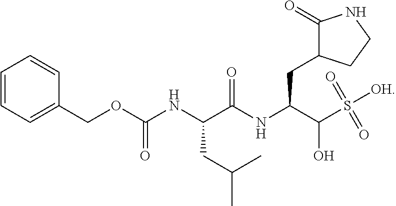 [SMILES: CC(C)C[C@H](NC(=O)OCc1ccccc1)C(=O)N[C@@H](CC1CCNC1=O)C(O)S(=O)(=O)O]

GRL-0617 refers to a compound of structure:

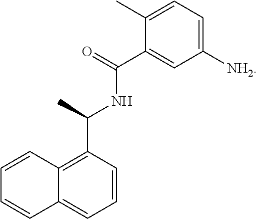 [SMILES: Cc1ccc(N)cc1C(=O)N[C@H](C)c1cccc2ccccc12]

In one aspect, provided herein is a bioluminescence resonance energy transfer (BRET) sensor comprising: 1) a bioluminescent protein, or mutants thereof; 2) a first BRET acceptor comprising a monomeric NeonGreen (mNG) fluorescent protein; and 3) a second BRET acceptor comprising a red fluorescent protein (RFP); wherein the sensor detects the activity of two proteins.

In some embodiments, the sensor detects the cleavage of two proteolytic enzymes. In some embodiments, the sensor detects the activity of two proteases.

In some embodiments, the bioluminescent protein is a luciferase, or an aequorin.

In some embodiments, the bioluminescent protein is luciferase. In some embodiments, a luciferase is selected from those found in Gaussia, Coleoptera, Renilla, Vargula, Oplophorus, mutants thereof, portions thereof, variants thereof, and/or any other luciferase enzymes suitable for the systems and methods described herein. In some embodiments, the bioluminescent reporter protein is a modified, enhanced luciferase enzyme from Oplophorus (e.g., NANOLUC enzyme from Promega Corporation, or a sequence with at least 70% identity (e.g., >70%, >80%, >90%, >95%) thereto). In some embodiments, the bioluminescent protein is a thermostable Photuris pennsylvanica luciferase or a sequence with at least 70% identity (e.g., >70%, >80%, >90%, >95%) thereto). Other bioluminescent proteins suitable for the sensors provided herein are described, for example, in US2010/0281552 andUS2012/0174242, both of which are herein incorporated by reference for all they disclose regarding bioluminescent proteins. In some embodiments, the bioluminescent protein is an artificial luciferase (ALuc) or a miniaturized variant thereof, named picALuc. In some embodiments, the bioluminescent protein is wild-type firefly-derived luciferase (FLuc), NanoLuc, TurboLuc, ALuc, picALuc, luciferase derived from Gaussia princeps (GLuc), luciferase derived from Renilla reniformis, or luciferase derived from Metridia longa (MLuc).

In some embodiments, a substrate for the bioluminescent protein is provided. In some embodiments, the bioluminescent protein converts the substrate into a reaction product and releases light energy as a by-product. In some embodiments, the substrate is a substrate for a luciferase enzyme, e.g., coelenterazine or a derivative thereof, or D-luciferin.

In some embodiments, the bioluminescent protein is selected from Renilla luciferase, firefly luciferase, Gaussia luciferase, or mutants thereof. In some embodiments, the bioluminescent protein is selected from a nano-luciferase (NLuc), or a pic-luciferase (picALuc).

In some embodiments of the sensor, the mNG fluorescent protein is attached to the N-terminal of the bioluminescent protein. In some embodiments of the sensor, the RFP is attached to the C-terminal of the bioluminescent protein.

In some embodiments of the sensor, the green fluorescent protein comprises a green fluorescent protein from Branchiostoma lanceolatum. Other green fluorescent proteins derived from, e.g., corals, sea anemones, zoanithids, copepods, and/or lancelets are also contemplated within the scope of embodiments presented herein. In some embodiments of the sensor, the RFP is selected from CyOFP1, mScarlet, LSS-mKate2, or mBeRFP tagged protein.

In some embodiments of the sensor, (i) the bioluminescent protein has an emission spectrum with a peak emission; (ii) the mNG fluorescent protein has a first excitation spectrum that overlaps with the emission spectrum; and (iii) the RFP has a second excitation spectrum that overlaps with the emission spectrum, wherein the first excitation spectrum is separated from the second excitation spectrum.

In some embodiments, the second excitation spectrum is separated from the first excitation spectrum by about 10 nm to about 100 nm. In some embodiments, the second excitation spectrum is separated from the first excitation spectrum by about 10 nm to about 50 nm. In some embodiments, the second excitation spectrum is separated from the first excitation spectrum by about 10 nm to about 30 nm. In some embodiments, the second excitation spectrum is separated from the first excitation spectrum by about 10 nm. In some embodiments, the second excitation spectrum is separated from the first excitation spectrum by at least about 10 nm, at least about 20 nm, at least about 30 nm, at least about 40 nm, or at least about 50 nm.

In some embodiments, the sensor detects proteolytic cleavage of two proteases. In some embodiments, the sensor detects proteolytic cleavage of coronavirus main protease (M^{pro}) and coronavirus papain-like protease (PL^{pro}). In some embodiments, the coronavirus is SARS-CoV-2. Any other pair of proteases (e.g., coronavirus proteases) is also contemplated within the scope of embodiments presented herein.

In some embodiments of the sensor, the mNG fluorescent protein is a mNG coronavirus M^{pro} protease, and the RFP is an RFP coronavirus PL^{pro} protease. In such embodiments, the reduction in fluorescence in the green channel indicates the activity of the M^{pro} protease, and a reduction in fluorescence in the red channel indicates the activity of the PL^{pro} protease.

Provided herein is a method for detecting a coronavirus infection in a patient, the method comprising:
(i) contacting a biological sample from the patient with a biosensor disclosed herein;
(ii) adding a substrate for the bioluminescent protein to the mixture; and
(iii) measuring fluorescence in a green channel and a red channel;
wherein
a reduction in the green channel fluorescence of the sensor indicates the presence of the coronavirus M^{pro} protease;
a reduction in the red channel fluorescence of the sensor indicates the presence of the coronavirus PL^{pro} protease; and
wherein the detection of coronavirus M^{pro} protease or coronavirus PL^{pro} protease, or both, indicates that the patient has a coronavirus infection. In some embodiments, the coronavirus is SARS-CoV-2.

Provided herein is a method for simultaneously testing inhibitors of coronavirus M^{pro} and coronavirus PL^{pro}, the method comprising:
(i) contacting a test compound with a mixture comprising coronavirus M^{pro} and coronavirus PL^{pro};
(ii) adding a dual protease sensor disclosed herein to the mixture;
(iii) adding a substrate for the bioluminescent protein to the mixture; and
(iii) measuring fluorescence in a green channel and a red channel.

In some embodiments of the method, the activity of a coronavirus M^{pro} protease results in the cleavage of the sensor resulting in a decrease in the BRET between the bioluminescent protein and the mNG protein, thereby reducing the green fluorescence of the sensor, and indicating the activity of the coronavirus M^{pro} protease and lack of inhibition by the test compound. If the test compound inhibits the coronavirus M^{pro} protease, the green fluorescence of the sensor is not be reduced. In some embodiments, the coronavirus is SARS-CoV-2.

In some embodiments of the method, the activity of a coronavirus PL^{pro} protease results in the cleavage of the sensor resulting in a decrease of BRET between the bioluminescent protein and the RFP, thereby reducing the red fluorescence of the sensor, and indicating the activity of the coronavirus PL^{pro} protease and lack of inhibition by the test compound. If the test compound inhibits the coronavirus PL^{pro} protease, the red fluorescence of the sensor is not be reduced. In some embodiments, the coronavirus is SARS-CoV-2.

Provided herein is a dual protease sensor for detecting a coronavirus in a subject, wherein the biosensor comprises an amino acid construct of SEQ ID NO: 1, SEQ ID NO: 3, SEQ ID NO: 5, or SEQ ID NO: 7. Provided herein is a dual protease sensor for detecting a coronavirus in a subject, wherein the biosensor comprises an amino acid construct of SEQ ID NO: 1. Provided herein is a dual protease sensor for detecting a coronavirus in a subject, wherein the biosensor comprises an amino acid construct of SEQ ID NO: 3. Provided herein is a dual protease sensor for detecting a coronavirus in a subject, wherein the biosensor comprises an amino acid construct of SEQ ID NO: 5. Provided herein is a dual protease sensor for detecting a coronavirus in a subject, wherein the biosensor comprises an amino acid construct of SEQ ID NO: 7. In some embodiments, the coronavirus is SARS-CoV-2.

Provided herein is a plasmid comprising a nucleotide construct of SEQ ID NO: 2, SEQ ID NO: 4, SEQ ID NO: 6, or SEQ ID NO: 8. Provided herein is a plasmid comprising a nucleotide construct of SEQ ID NO: 2. Provided herein is a plasmid comprising a nucleotide construct of SEQ ID NO: 4. Provided herein is a plasmid comprising a nucleotide construct of SEQ ID NO: 6. Provided herein is a plasmid comprising a nucleotide construct of SEQ ID NO: 8.

Provided herein is a cell transfected with a plasmid comprising a nucleotide construct of SEQ ID NO: 2, SEQ ID NO: 4, SEQ ID NO: 6, or SEQ ID NO: 8. Provided herein is a cell transfected with a plasmid comprising a nucleotide construct of SEQ ID NO: 2. Provided herein is a cell transfected with a plasmid comprising a nucleotide construct of SEQ ID NO: 4. Provided herein is a cell transfected with a plasmid comprising a nucleotide construct of SEQ ID NO: 6. Provided herein is a cell transfected with a plasmid comprising a nucleotide construct of SEQ ID NO: 8.

Also provided is a kit comprising the sensor described herein, and a substrate for the bioluminescent protein.

In some embodiments, provided is a biosensor for detecting proteolytic cleavage activity of two proteases, coronavirus M^{pro} and PL^{pro}. In some embodiments, the biosensor utilizes BRET between a donor and two acceptor moieties. In some embodiments, the biosensor utilizes different acceptor fluorescent proteins in green and red channels.

BRET-based coronavirus dual protease biosensor construct's amino acid and nucleotide sequence details are provided below.

mNG-Mpro-Nter-auto-NLuc-PLpro-Nter-auto-mBeRFP:
Amino Acid Sequence
[SEQ ID NO: 1]
MGSSHHHHHHSSGLVPRGSHMVSKGEEDNMASLPATHELHIFGSINGVDEDMVGQGTGNPNDGYEELNLK
STKGDLQFSPWILVPHIGYGFHQYLPYPDGMSPFQAAMVDGSGYQVHRTMQFEDGASLTVNYRYTYEGSH
IKGEAQVKGTGFPADGPVMTNSLTAADWCRSKKTYPNDKTIISTFKWSYTTGNGKRYRSTARTTYTFAKP
MAANYLKNQPMYVFRKTELKHSKTELNFKEWQKAFTDVMGMDELYKAAATENLYAVLQSGFRGSGSAMVF
TLEDFVGDWRQTAGYNLDQVLEQGGVSSLFQNLGVSVTPIQRIVLSGENGLKIDIHVIIPYEGLSGDQMG
QIEKIFKVVYPVDDHHFKVILHYGTLVIDGVTPNMIDYFGRPYEGIAVEDGKKITVTGTLWNGNKIIDER
LINPDGSLLFRVTINGVTGWRLCERILAGPRSGSLKGGAPTKASQEFMVSKGEELIKENMHMKLYMEGTV
NNHHFKCTSEGEGKPYEGTQTMRIKVVEGGPLPFAFDILATSFMYGSKTFINHTQGIPDFFKQSFPEGFT
WERSTTYEDGGVLTATQDTSLQDGCLIYNVKIRGVNFPSNGPVMQKKTLGWEASTEMLYPADGGLEGRDY
MALKLVGGGHLICNAKTTYRSKKPAKNLKMPGVYYVDRRLERIKEADKETSVEQHEVAVARYCDLPSKLG
HKGTDYKDHDGDYKDHDIDYKDDDDKDI*
Nucleotide Sequence
[SEQ ID NO: 2]
atgggaagttcacatcatcatcatcatcactcatcaggactggtgccacgggggtctcacatggtctcca
agggggaagaggacaacatggcctctctgcctgccacacacgagctgcacatcttcggcagcatcaatgg
cgtggactttgatatggtgggccagggcacaggcaacccaaatgacggctacgaggagctgaacctgaag
agcaccaagggcgatctgcagttctccccttggatcctggtgccacacatcggctatggctttcaccagt
atctgccctaccctgacggcatgtcccccttccaggccgccatggtggatggctctggctaccaggtgca
caggaccatgcagtttgaggacggcgcctccctgacagtgaattaccggtatacctacgagggctctcac
atcaagggcgaggcccaggtgaagggcacaggcttcccagccgatggccccgtgatgacaaactctctga
ccgccgccgactggtgccggagcaagaagacctaccctaatgataagacaatcatctccacctttaagtg
gtcttataccacaggcaacggcaagcggtacagaagcacagccagaaccacatatacctttgccaagcca
atggccgccaactatctgaagaatcagcccatgtacgtgttcaggaagacagagctgaagcactccaaga
ccgagctgaatttcaaggagtggcagaaggcctttacagacgtgatgggcatggatgagctgtacaaggg
cggccgccaccgagaacctgtatgcagtgctccaaagcggatttcgcggctctggcagcgctatggtgtt
cacactggaggactttgtgggcgattggcggcagaccgccggctataatctggatcaggtgctggagcag
ggcggcgtgagctccctgttccagaacctgggcgtgagcgtgacccctatccagcggatcgtgctgtccg
gcgagaacggcctgaagatcgacatccacgtgatcatcccatacgagggcctgtctggcgatcagatggg
ccagatcgagaagatcttcaaggtggtgtaccccgtggacgatcaccacttcaaagtgatcctgcactat
ggcaccctggtcatcgacggcgtgacccccaatatgatcgattatttcggccggccttacgagggcatcg
ccgtgtttgacggcaagaagatcaccgtgacaggcaccctgtggaacggcaataagatcatcgacgagag
actgatcaaccccgatggctccctgctgttcagggtgactattaacggagtgactggctggcggctgtgc
gaaaggattctggctggaccgcggagcgggagcctgaaaggcggcgcgccgaccaaagcgagtcaggaat
tcatggtgtccaaaggcgaagaactgattaaagaaaatatgcatatgaaactgtatatggaaggcaccgt
gaataatcatcattttaaatgcacctccgaaggcgaaggcaaaccgtatgaaggcacccagaccatgcgt
attaaagtggtggaaggcggcccgctgccgtttgcgtttgatattctggcgacctcctttatgtatggct
ccaaaacctttattaatcatacccagggcattccggatttttttaaacagtcctttccggaaggctttac
ctgggaacgttccaccacctatgaagatggcggcgtgctgaccgcgacccaggatacctccctgcaggat
ggctgcctgatttataatgtgaaaattcgtggcgtgaattttccgtccaatggcccggtgatgcagaaaa
aaaccctgggctgggaagcgtccaccgaaatgctgtatccggcggatggcggcctggaaggccgtgatta
tatggcgctgaaactggtgggcggcggccatctgatttgcaatgcgaaaaccacctatcgttccaaaaaa
ccggcgaaaaatctgaaaatgccgggcgtgtattatgtggatcgtcgtctggaacgtattaaagaagcgg
ataaagaaacctccgtggaacagcatgaagtggcggtggcgcgttattgcgatctgccgtccaaactggg
ccataaaggtaccgactacaaagaccatgacggtgattataaagatcatgacatcgattacaaggatgac
gatgacaaggatatctga*
Theoretical pI: 6.26; Theoretical MW: 81.3 kDa
mNG-Mpro-Nter-auto-NLuc-PLpro-Nter-auto-LSS-mKate2:
Amino Acid Sequence
[SEQ ID NO: 3]
MGSSHHHHHHSSGLVPRGSHMVSKGEEDNMASLPATHELHIFGSINGVDEDMVGQGTGNPNDGYEELNLK
STKGDLQFSPWILVPHIGYGFHQYLPYPDGMSPFQAAMVDGSGYQVHRTMQFEDGASLTVNYRYTYEGSH
IKGEAQVKGTGFPADGPVMTNSLTAADWCRSKKTYPNDKTIISTFKWSYTTGNGKRYRSTARTTYTFAKP
MAANYLKNQPMYVFRKTELKHSKTELNFKEWQKAFTDVMGMDELYKAAATENLYAVLQSGFRGSGSAMVF
TLEDFVGDWRQTAGYNLDQVLEQGGVSSLFQNLGVSVTPIQRIVLSGENGLKIDIHVIIPYEGLSGDQMG
QIEKIFKVVYPVDDHHFKVILHYGTLVIDGVTPNMIDYFGRPYEGIAVFDGKKITVTGTLWNGNKIIDER
LINPDGSLLFRVTINGVTGWRLCERILAGPRSGSLKGGAPTKASQEFMVSKGEELIKENMHMKLYMEGTV
NNHHFKCTSEGEGKPYEGTQTMRIKVVEGGPLPFAFDILATSEMYGSYTFINHTQGIPDFFKQSFPEGFT
WERVTTYEDGGVLTATQDTSLQDGCLIYNVKIRGVNFTSNGPVMQKKTLGWEAGTEMLYPADGGLEGRSD
DALKLVGGGHLICNLKSTYRSKKPAKNLKVPGVYYVDRRLERIKEADKETYVEQHEVAVARYCDLPSKLG
HKLNGTDYKDHDGDYKDHDIDYKDDDDKDI
Nucleotide Sequence
[SEQ ID NO: 4]
atgggaagttcacatcatcatcatcatcactcatcaggactggtgccacgggggtctcacatggtctcca
agggggaagaggacaacatggcctctctgcctgccacacacgagctgcacatcttcggcagcatcaatgg
cgtggactttgatatggtgggccagggcacaggcaacccaaatgacggctacgaggagctgaacctgaag
agcaccaagggcgatctgcagttctccccttggatcctggtgccacacatcggctatggctttcaccagt
atctgccctaccctgacggcatgtcccccttccaggccgccatggtggatggctctggctaccaggtgca
caggaccatgcagtttgaggacggcgcctccctgacagtgaattaccggtatacctacgagggctctcac
atcaagggcgaggcccaggtgaagggcacaggcttcccagccgatggccccgtgatgacaaactctctga
ccgccgccgactggtgccggagcaagaagacctaccctaatgataagacaatcatctccacctttaagtg
gtcttataccacaggcaacggcaagcggtacagaagcacagccagaaccacatatacctttgccaagcca
atggccgccaactatctgaagaatcagcccatgtacgtgttcaggaagacagagctgaagcactccaaga
ccgagctgaatttcaaggagtggcagaaggcctttacagacgtgatgggcatggatgagctgtacaaggc
ggccgccaccgagaacctgtatgcagtgctccaaagcggatttcgcggctctggcagcgctatggtgttc
acactggaggactttgtgggcgattggcggcagaccgccggctataatctggatcaggtgctggagcagg
gcggcgtgagctccctgttccagaacctgggcgtgagcgtgacccctatccagcggatcgtgctgtccgg
cgagaacggcctgaagatcgacatccacgtgatcatcccatacgagggcctgtctggcgatcagatgggc
cagatcgagaagatcttcaaggtggtgtaccccgtggacgatcaccacttcaaagtgatcctgcactatg
gcaccctggtcatcgacggcgtgacccccaatatgatcgattatttcggccggccttacgagggcatcgc
cgtgtttgacggcaagaagatcaccgtgacaggcaccctgtggaacggcaataagatcatcgacgagaga
ctgatcaaccccgatggctccctgctgttcagggtgactattaacggagtgactggctggcggctgtgcg
aaaggattctggctggaccgcggagcgggagcctgaaaggcggcgcgccgaccaaagcgagtcaggaatt
catggtgtctaagggcgaagagctgattaaggagaacatgcacatgaagctgtacatggaaggcaccgtg
aacaaccaccacttcaagtgcacatccgagggcgaaggcaagccctacgagggcacccagaccatgagaa
tcaaggtggtcgagggcggccctctacccttcgccttcgacatcttggctaccagcttcatgtacggcag
ctacaccttcatcaaccacacccagggcatccccgacttctttaagcagtccttccctgagggcttcaca
tgggagagagtcaccacatacgaagacgggggcgtgctgaccgctacccaggacaccagcctccaggacg
gttgcctcatctacaacgtcaagatcagaggggtgaacttcacatccaacggccctgtgatgcagaagaa
aacactcggctgggaggccggcaccgagatgctgtaccccgctgacggcggcctggaaggcagatcggac
gacgccctgaagctcgtgggcgggggccacctgatctgcaacttgaagagcacatacagatccaagaaac
ccgctaagaatctcaaggtgcccggcgtctactatgtggaccgaagactggaaagaatcaaggaggccga
caaagagacctacgtcgagcagcacgaggtggctgtggccagatactgcgacctccctagcaaactgggg
cacaagcttaatggtaccgactacaaagaccatgacggtgattataaagatcatgacatcgattacaagg
atgacgatgacaaggatatctga*
Theoretical pI: 6.15; Theoretical MW: 81.5 kDa
mNG-Mpro-Nter-auto-NLuc-PLpro-Nter-auto-LSS-CyOFP1:
Amino Acid Sequence
[SEQ ID NO: 5]
MGSSHHHHHHSSGLVPRGSHMVSKGEEDNMASLPATHELHIFGSINGVDFDMVGQGTGNPNDGYEELNLK
STKGDLQFSPWILVPHIGYGFHQYLPYPDGMSPFQAAMVDGSGYQVHRTMQFEDGASLTVNYRYTYEGSH
IKGEAQVKGTGFPADGPVMTNSLTAADWCRSKKTYPNDKTIISTFKWSYTTGNGKRYRSTARTTYTFAKP
MAANYLKNQPMYVFRKTELKHSKTELNFKEWQKAFTDVMGMDELYKAAATENLYAVLQSGFRGSGSAMVF
TLEDFVGDWRQTAGYNLDQVLEQGGVSSLFQNLGVSVTPIQRIVLSGENGLKIDIHVIIPYEGLSGDQMG
QIEKIFKVVYPVDDHHFKVILHYGTLVIDGVTPNMIDYFGRPYEGIAVEDGKKITVTGTLWNGNKIIDER
LINPDGSLLFRVTINGVTGWRLCERILAGPRSGSLKGGAPTKASQEFMVSKGEELIKENMRSKLYLEGSV
NGHQFKCTHEGEGKPYEGKQTNRIKVVEGGPLPFAFDILATHEMYGSKVFIKYPADLPDYFKQSFPEGFT
WERVMVFEDGGVLTATQDTSLQDGELIYNVKVRGVNFPANGPVMQKKTLGWEPSTETMYPADGGLEGRCD
KALKLVGGGHLHVNFKTTYKSKKPVKMPGVHYVDRRLERIKEADNETYVEQYEHAVARYSNLGGGFTLED
FVGTDYKDHDGDYKDHDIDYKDDDDKDI*
Nucleotide Sequence
[SEQ ID NO: 6]
atgggaagttcacatcatcatcatcatcactcatcaggactggtgccacgggggtctcacatggtctcca
agggggaagaggacaacatggcctctctgcctgccacacacgagctgcacatcttcggcagcatcaatgg
cgtggactttgatatggtgggccagggcacaggcaacccaaatgacggctacgaggagctgaacctgaag
agcaccaagggcgatctgcagttctccccttggatcctggtgccacacatcggctatggctttcaccagt
atctgccctaccctgacggcatgtcccccttccaggccgccatggtggatggctctggctaccaggtgca
caggaccatgcagtttgaggacggcgcctccctgacagtgaattaccggtatacctacgagggctctcac
atcaagggcgaggcccaggtgaagggcacaggcttcccagccgatggccccgtgatgacaaactctctga
ccgccgccgactggtgccggagcaagaagacctaccctaatgataagacaatcatctccacctttaagtg
gtcttataccacaggcaacggcaagcggtacagaagcacagccagaaccacatatacctttgccaagcca
atggccgccaactatctgaagaatcagcccatgtacgtgttcaggaagacagagctgaagcactccaaga
ccgagctgaatttcaaggagtggcagaaggcctttacagacgtgatgggcatggatgagctgtacaaggc
ggccgccaccgagaacctgtatgcagtgctccaaagcggatttcgcggctctggcagcgctatggtgttc
acactggaggactttgtgggcgattggcggcagaccgccggctataatctggatcaggtgctggagcagg
gcggcgtgagctccctgttccagaacctgggcgtgagcgtgacccctatccagcggatcgtgctgtccgg
cgagaacggcctgaagatcgacatccacgtgatcatcccatacgagggcctgtctggcgatcagatgggc
cagatcgagaagatcttcaaggtggtgtaccccgtggacgatcaccacttcaaagtgatcctgcactatg
gcaccctggtcatcgacggcgtgacccccaatatgatcgattatttcggccggccttacgagggcatcgc
cgtgtttgacggcaagaagatcaccgtgacaggcaccctgtggaacggcaataagatcatcgacgagaga
ctgatcaaccccgatggctccctgctgttcagggtgactattaacggagtgactggctggcggctgtgcg
aaaggattctggctggaccgcggagcgggagcctgaaaggcggcgcgccgaccaaagcgagtcaggaatt
catggtgagcaagggcgaggagctgatcaaggagaacatgagaagcaagctgtacctggaaggcagcgtg
aacggccaccagttcaagtgcacccacgaaggggagggcaagccctacgagggcaagcagaccaacagga
tcaaggtggtggagggaggccccctgccgttcgcattcgacatcctggccacccactttatgtacgggag
caaggtgttcatcaagtaccccgccgacctccccgattattttaagcagtccttccctgagggcttcaca
tgggagagagtcatggtgttcgaagacgggggcgtgctgaccgccacccaggacaccagcctccaggacg
gcgagctcatctacaacgtcaaggtcagaggggtgaacttcccagccaacggccccgtgatgcagaagaa
aacactgggctgggagcccagcaccgagaccatgtaccccgctgacggcggcctggaaggcagatgcgac
aaggccctgaagctcgtgggcgggggccacctgcacgtcaacttcaagaccacatacaagtccaagaaac
ccgtgaagatgcccggcgtccactacgtggaccgcagactggaaagaatcaaggaggccgacaacgagac
ctacgtcgagcagtacgagcacgctgtggccagatactccaacctgggcggaggcttcacactcgaagat
ttcgttggtaccgactacaaagaccatgacggtgattataaagatcatgacatcgattacaaggatgacg
atgacaaggatatctga*
Theoretical pI: 6.09; Theoretical Mw: 81.4 kDa
mNG-Mpro-Nter-auto-NLuc-PLpro-Nter-auto-mScarlet:
Amino Acid Sequence
[SEQ ID NO: 7]
MGSSHHHHHHSSGLVPRGSHMVSKGEEDNMASLPATHELHIFGSINGVDFDMVGQGTGNPNDGYEELNLK
STKGDLQFSPWILVPHIGYGFHQYLPYPDGMSPFQAAMVDGSGYQVHRTMQFEDGASLTVNYRYTYEGSH
IKGEAQVKGTGFPADGPVMTNSLTAADWCRSKKTYPNDKTIISTFKWSYTTGNGKRYRSTARTTYTFAKP
MAANYLKNQPMYVFRKTELKHSKTELNFKEWQKAFTDVMGMDELYKAAATENLYAVLQSGFRGSGSAMVF
TLEDFVGDWRQTAGYNLDQVLEQGGVSSLFQNLGVSVTPIQRIVLSGENGLKIDIHVIIPYEGLSGDQMG
QIEKIFKVVYPVDDHHFKVILHYGTLVIDGVTPNMIDYFGRPYEGIAVEDGKKITVTGTLWNGNKIIDER
LINPDGSLLFRVTINGVTGWRLCERILAGPRSGSLKGGAPTKASQEFMVSKGEAVIKEFMRFKVHMEGSM
NGHEFEIEGEGEGRPYEGTQTAKLKVTKGGPLPFSWDILSPQFMYGSRAFTKHPADIPDYYKQSFPEGFK
WERVMNFEDGGAVTVTQDTSLEDGTLIYKVKLRGTNFPPDGPVMQKKTMGWEASTERLYPEDGVLKGDIK
MALRLKDGGRYLADFKTTYKAKKPVQMPGAYNVDRKLDITSHNEDYTVVEQYERSEGRHSTGGMDELYKG
TDYKDHDGDYKDHDIDYKDDDDKDI*
Nucleotide Sequence
[SEQ ID NO: 8]
atgggaagttcacatcatcatcatcatcactcatcaggactggtgccacgggggtctcacatggtctcca
agggggaagaggacaacatggcctctctgcctgccacacacgagctgcacatcttcggcagcatcaatgg
cgtggactttgatatggtgggccagggcacaggcaacccaaatgacggctacgaggagctgaacctgaag
agcaccaagggcgatctgcagttctccccttggatcctggtgccacacatcggctatggctttcaccagt
atctgccctaccctgacggcatgtcccccttccaggccgccatggtggatggctctggctaccaggtgca
caggaccatgcagtttgaggacggcgcctccctgacagtgaattaccggtatacctacgagggctctcac
atcaagggcgaggcccaggtgaagggcacaggcttcccagccgatggccccgtgatgacaaactctctga
ccgccgccgactggtgccggagcaagaagacctaccctaatgataagacaatcatctccacctttaagtg
gtcttataccacaggcaacggcaagcggtacagaagcacagccagaaccacatatacctttgccaagcca
atggccgccaactatctgaagaatcagcccatgtacgtgttcaggaagacagagctgaagcactccaaga
ccgagctgaatttcaaggagtggcagaaggcctttacagacgtgatgggcatggatgagctgtacaaggc
ggccgccaccgagaacctgtatgcagtgctccaaagcggatttcgcggctctggcagcgctatggtgttc
acactggaggactttgtgggcgattggcggcagaccgccggctataatctggatcaggtgctggagcagg
gcggcgtgagctccctgttccagaacctgggcgtgagcgtgacccctatccagcggatcgtgctgtccgg
cgagaacggcctgaagatcgacatccacgtgatcatcccatacgagggcctgtctggcgatcagatgggc
cagatcgagaagatcttcaaggtggtgtaccccgtggacgatcaccacttcaaagtgatcctgcactatg
gcaccctggtcatcgacggcgtgacccccaatatgatcgattatttcggccggccttacgagggcatcgc
cgtgtttgacggcaagaagatcaccgtgacaggcaccctgtggaacggcaataagatcatcgacgagaga
ctgatcaaccccgatggctccctgctgttcagggtgactattaacggagtgactggctggcggctgtgcg
aaaggattctggctggaccgcggagcgggagcctgaaaggcggcgcgccgaccaaagcgagtcaggaatt
catggtgagcaagggcgaggcagtgatcaaggagttcatgcggttcaaggtgcacatggagggctccatg
aacggccacgagttcgagatcgagggcgagggcgagggccgcccctacgagggcacccagaccgccaagc
tgaaggtgaccaagggtggccccctgcccttctcctgggacatcctgtcccctcagttcatgtacggctc
cagggccttcaccaagcaccccgccgacatccccgactactataagcagtccttccccgagggcttcaag
tgggagcgcgtgatgaacttcgaggacggcggcgccgtgaccgtgacccaggacacctccctggaggacg
gcaccctgatctacaaggtgaagctccgcggcaccaacttccctcctgacggccccgtaatgcagaagaa
gacaatgggctgggaagcgtccaccgagcggttgtaccccgaggacggcgtgctgaagggcgacattaag
atggccctgcgcctgaaggacggcggccgctacctggcggacttcaagaccacctacaaggccaagaagc
ccgtgcagatgcccggcgcctacaacgtcgaccgcaagttggacatcacctcccacaacgaggactacac
cgtggtggaacagtacgaacgctccgagggccgccactccaccggcggcatggacgagctgtacaagggt
accgactacaaagaccatgacggtgattataaagatcatgacatcgattacaaggatgacgatgacaagg
atatctga*
Theoretical pI: 5.99; Theoretical Mw: 81.2 kDa

### EXAMPLES

### Example 1

### Materials and Methods

### BRET-Based Dual Protease Biosensor (DuProSense) Plasmid Construct Generation

We generated four BRET-based dual protease biosensor (DuProSense) constructs containing mNG at the N-terminal and mBeRFP, LSS-mKate2, CyOFP1, or mScarlet RFPs at the C-terminal of NLuc luciferase protein. The dual protease biosensor constructs were developed based on previously reported M^{pro} N-terminal autocleavage peptide sequence, AVLQSGFR SEQ ID NO: 9 (nucleotide sequence 5′ GCA GTG CTC CAA AGC GGA TTT CGC 3′ SEQ ID NO: 10) and PL^{pro} N-terminal autocleavage peptide sequence, LKGGAPTK SEQ ID NO: 11 (nucleotide sequence 5′ CTG AAA GGC GGC GCG CCG ACC AAA 3′ SEQ ID NO: 12). Initially, a gene fragment mNG-M^{pro}-NLuc-PL^{pro}-mBeRFP containing a part of the mNG coding sequence and BstXI and Xbal restriction enzyme sites at the 5′ and 3′ termini, respectively, was synthesized (Integrated DNA Technologies) and introduced into the pUC57 plasmid to generate the pUC57-mNG-M^{pro}-NLuc-PL^{pro}-BeRFP plasmid construct. The DNA fragment, mNG-M^{pro}-NLuc-PL^{pro}-mBeRFP was excised from the pUC57-mNG-M^{pro}-NLuc-PL^{pro}-BeRFP plasmid using the restriction enzymes BstXI and Xbal and ligated to the similarly digested mNeonGreen-DEVD-nanoLuc plasmid (Addgene: #98287) to generate the pmNG-M^{pro}-NLuc-PL^{pro}-mBeRFP (DuProSense with mBeRFP as the RFP) plasmid. Subsequently, LSS-mKate2, CyOFP1 and mScarlet gene fragments were PCR amplified from pLSSmKate2-C1 (Addgene #31869), pcDNA3-Antares2 c-myc (Addgene #100027) and pmScarlet_C1 (Addgene #85042) plasmids, respectively, using forward primers containing an EcoRI restriction enzyme site and reverse primers containing Kpnl restriction enzyme. The PCR products and the pmNG-M^{pro}-NLuc-PL^{pro}-mBeRFP plasmid was digested using EcoRI and Kpnl restriction enzymes and ligated using T4 DNA ligase (ThermoFisher Scientific). The clones pmNG-M^{pro}-NLuc-PL^{pro}-LSS-mKate2, pmNG-M^{pro}-NLuc-PL^{pro}-CYOFP1, and pmNG-M^{pro}-NLuc-PL^{pro}-mScarlet were confirmed by restriction-digestion. All plasmid sequences were further confirmed by Sanger sequencing (Macrogen) using CMV forward primer and respective reverse primers. All four DuProSense constructs contained a 6×His-tag at the N-terminal and 3×-FLAG-tag at the C-terminal.

The M^{pro} cleavage site mutant of the mScarlet-based DuProSense (pmNG-M^{pro}-mut-NLuc-PL^{pro}-mScarlet) was generated by substituting Gln to Ala at the P1 site in the autocleavage peptide sequence of M^{pro} (AVLASGFR SEQ ID NO: 13; nucleotide sequence 5′ GCA GTG CTC GCC AGC GGA TTT CGC 3′ SEQ ID NO: 14). The PL^{pro} cleavage site mutant of the mScarlet-based DuProSense (pmNG-M^{pro}-NLuc-PL^{pro}-mut-mScarlet) was generated by replacing Gly to IIe in the PL^{pro} cleavage site (LKGIAPTK SEQ ID NO: 15; nucleotide sequence 5′ CTG AAA GGC ATT GCG CCG ACC AAA SEQ ID NO: 16).

### Cell Culture and Transfection

HEK293T cells were used to perform both live cells and in vitro assays. This cell line was grown in Dulbecco's Modified Eagle Medium (DMEM) supplemented with 10% fetal bovine serum, and 1% penicillin-streptomycin at 37° C. in 5% CO2. For live cell assay, HEK293T cells seeded onto 96-well white plates 24 h prior were used for polyethyleneimine (PEI) lipid-mediated transfection. The plasmid DNA (at a 1:5 ratio of either DuProSense biosensor or the mutant sensor plasmid DNA to protease, either M^{pro} or PL^{pro} or both, plasmid DNA) and Opti-MEM (Invitrogen) were mixed using pipetting and brief vortex after adding 1.25 μg/well of PEI lipid (Sigma-Aldrich). The transfection mix was incubated at room temperature for 30 min before adding to wells drop by drop. For determining the effect of GC376 and GRL-0617, M^{pro} and PL^{pro} protease pharmacological inhibitors, respectively, cells were co-transfected with the mScarlet-based DuProSense and both M^{pro} and PL^{pro} protease plasmid DNA.

### Cell Lysate Preparation

HEK293T cells were seeded on 10 cm dishes one day prior to transfection. The cells in each 10 cm dish were transfected with one of the dual sensor plasmid constructs using PEI lipid. For red fluorescent protein control groups, cells were transfected with either pLSSmKate2-C1, pcDNA3-Antares2 c-myc, or pmScarlet_C1. The cell lysates were collected after 48 h post-transfection by carefully removing the media from plates and proceeded to wash the cells with ice-cold Dulbecco's Phosphate-Buffered Saline (DBPS). After washing, 400 μL of chilled lysis buffer (50 mM HEPES (pH 7.5), 50 mM NaCl, 0.1% Triton-X 100, 1 mM dithiothreitol (DTT), and 1 mM ethylenediamine tetra acetic acid (EDTA)) was added to cells on ice. The mixture was collected into 1.5 mL eppendorf tube and centrifuged at 14,000 rotation per minute (rpm) for 1 h. After centrifugation, supernatant was collected and stored at −80° C. until further usage.

### Bioluminescence and Fluorescence Measurements

Live cell BRET and in vitro fluorescence measurements were performed using a Tecan SPARK® multimode microplate reader. Bioluminescence spectral scans were acquired as previously described (Commun Chem 5:117, 2022). For in vitro fluorescent spectral analysis, equal volume of cell lysates prepared from cells expressing the four DuProSense constructs (mBeRFP, LSS-mKate2, CyOFP1 and mScarlet) were excited at 440 nm and fluorescence spectra was measured.

### Live Cell Dual-Color BRET Measurements

HEK293T cells were co-transfected individually with the four DuProSense biosensor plasmids along with either pLVX-EF1alpha-SARS-CoV-2-nsp5-2xStrep-IRES-Puro (M^{pro} WT) (Addgene plasmid #141370) or Nsp3-EGFP (PL^{pro} WT) (Addgene plasmid #165108) plasmid or with both in 96-well white flat bottom plates at a ratio of 1:5 of biosensor-to-protease plasmid DNA ratio. After 48 h post-transfection, BRET measurements were performed by the addition of furimazine (Promega) at a dilution of 1:200. Three independent experiments were performed in triplicates for each biosensor construct. The formula for calculating BRET M^{pro} and BRET PL^{pro} is shown below.

$${BRET}_{Mpro}^{mBeRFP} = \frac{{I}_{533} + {I}_{632}}{{I}_{467}}$$

$${BRET}_{PLpro}^{mBeRFP} = \frac{{I}_{632}}{{I}_{467} + {I}_{533} + {I}_{566}}$$

$${BRET}_{Mpro}^{LSSmKate 2} = \frac{{I}_{533} + {I}_{615}}{{I}_{467}}$$

$${BRET}_{PLpro}^{LSSmKate 2} = \frac{{I}_{615}}{{I}_{467} + {I}_{533} + {I}_{566}}$$

$${BRET}_{Mpro}^{CyOFP 1} = \frac{{I}_{533} + {I}_{599}}{{I}_{467}}$$

$${BRET}_{PLpro}^{CyOFP 1} = \frac{{I}_{599}}{{I}_{467} + {I}_{533} + {I}_{566}}$$

$${BRET}_{Mpro}^{mScarlet} = \frac{{I}_{533} + {I}_{615}}{{I}_{467}}$$

$${BRET}_{Mpro}^{mScarlet} = \frac{{I}_{615}}{{I}_{467} + {I}_{533} + {I}_{566}}$$

I₅₃₃, I₆₃₂, I₆₁₅, I₅₉₉ and I₄₆₇ are relative intensities obtained from bioluminescence spectra of the indicated DuProSense biosensor construct.

Live Cell Inhibition of M^{pro} F and PL^{pro} by GC376 and GRL-0617

HEK293T cells were co-transfected with DuProSense-mScarlet plasmid and either pLVX-EF1alpha-SARS-CoV-2-nsp5-2xStrep-IRES-Puro (M^{pro} WT) or Nsp3-EGFP (PL^{pro} WT)) plasmid in 96-well white flat bottom plates. The ratio of biosensor-to-protease plasmid DNA was maintained at 1:5. To check the utility of DuProSense as an inhibitory assay reporter, transfected cells were simultaneously treated with a range of concentrations of corresponding protease inhibitor (GC376 against M^{pro}, GRL-0617 against PL^{pro}; both inhibitor stock solutions in 50% DMSO). After 20 hours of incubation with the inhibitor, BRET measurements were taken by adding furimazine at a dilution of 1:200. The percentage protease activity was calculated by normalizing the BRET values with negative control (in the absence of protease) and positive control (with the treatment of compound having no inhibitory effect on protease; GRL-0617 on M^{pro}, GC376 on PL^{pro}).

$$Per centage Inhibition = \frac{( Negative control BRET - Experimental BRET )}{( Negative control BRET - Positive control BRET )} \times 100$$

### Western Blot

HEK293T cells co-transfected with the DuProSense and the M^{pro} or PL^{pro} or both plasmids were lysed in 200 μL of 2× Laemmli sample buffer (50 mM Tris-Cl pH 6.8, 1.6% SDS, 8% glycerol, 4% β-mercaptoethanol, and 0.04% bromophenol blue) heated to 85° C. and sonicated prior to addition. Equal volumes of the cell lysates (30 μL) were separated by 10% SDS-PAGE followed by transferring proteins onto PVDF membranes. Membranes were blocked in Tris-buffered saline containing 0.1% Tween-20 (TBS-T) with bovine serum albumin (BSA-5%) for 1 h at room temperature. Blots were incubated with anti-flag antibody (DYKDDDDK SEQ ID NO: 17 Tag Mouse Monoclonal Antibody (FG4R); ThermoFisher Scientific) for overnight at 4° C. in dilution buffer (TBS-T containing 5% bovine serum albumin (BSA). Secondary anti-mouse IgG HRP (Anti-Mouse Ig:HRP Donkey pAb; ECM biosciences; 1:10000 diluted in TBS-T) was used to detect the cleaved biosensor proteins.

### Data Analysis

GraphPad Prism (version 9 for macOS, GraphPad Software), in combination with Microsoft Excel, was used for data analysis and graph preparation.

### Results

### Designing of a BRET-Based, Dual Protease Biosensor (DuProSense)

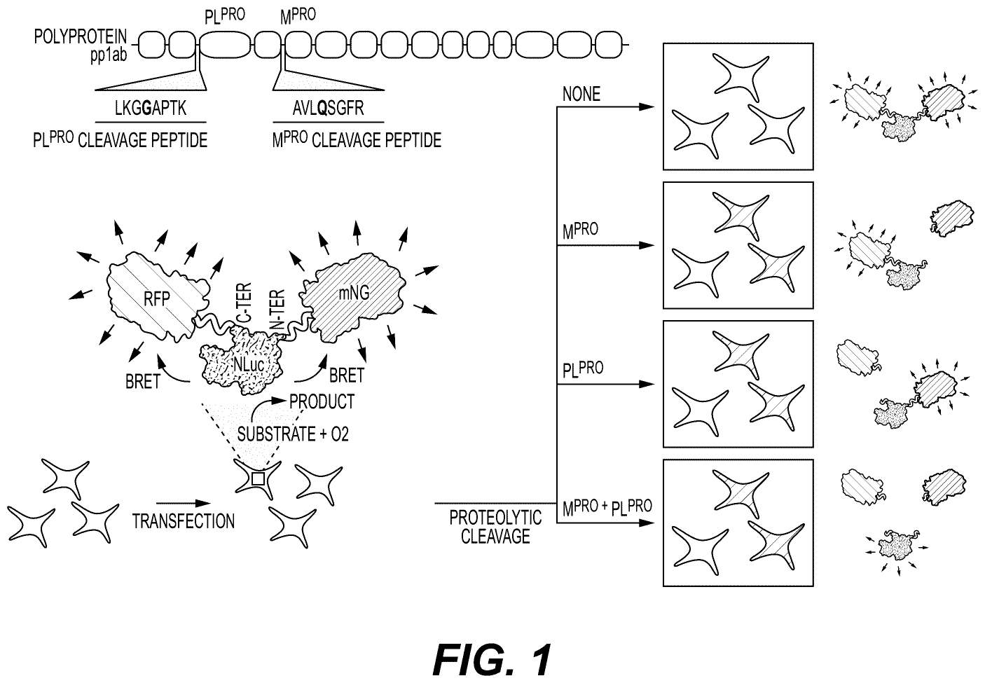

To increase the capability of BRET-based biosensors from detecting one molecular event to two molecular events, we aimed to develop a dual protease biosensor, DuProSense, to simultaneously detect two molecular events, i.e., proteolytic cleavage, using a single BRET-based biosensor (FIG. 1). For this, we engineered a fusion protein containing the monomeric mNG fluorescent protein, which acts as the BRET acceptor in the green channel, on the N-terminal of NLuc and a red fluorescent protein (RFP) on the C-terminal side of NLuc as the second BRET acceptor. Use of an RFP allowed unambiguous spectral separation of fluorescence signal from the second BRET acceptor. However, a challenge in this regard is the requirement for an overlap with bioluminescent donor emission spectrum and fluorescent acceptor protein excitation spectra. Additional factors we considered for our design include molecular brightness and maturation time. For this, we analyzed currently available RFPs in the literature (data available at fpbase.org) and chose to consider mBeRFP, LSS-mKate2, CyOFP1 and mScarlet RFPs (Table 1).

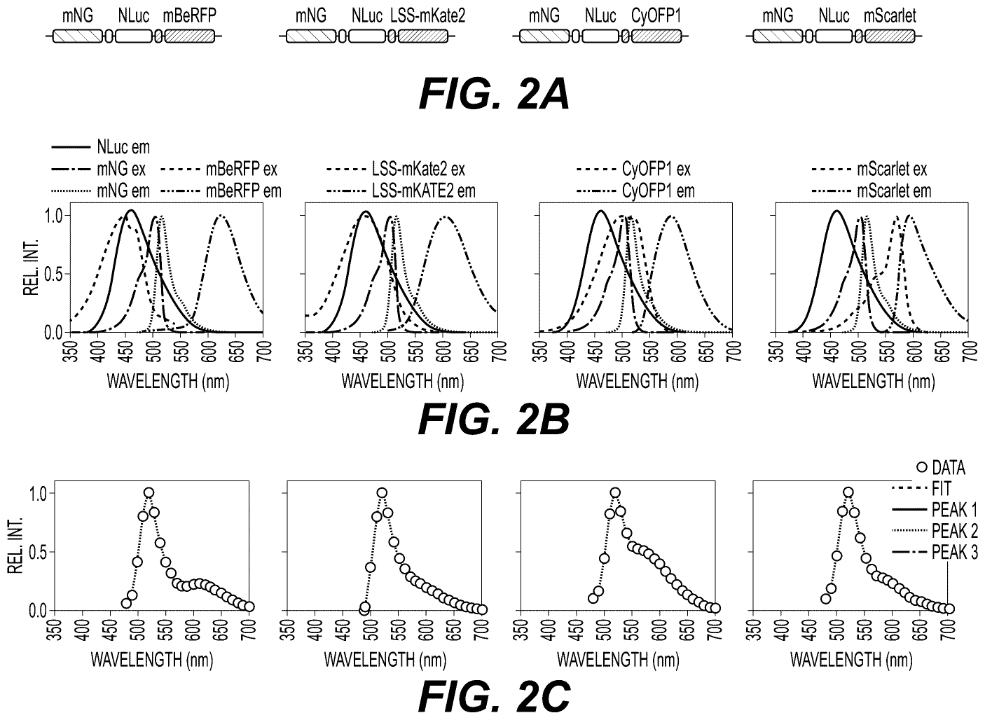

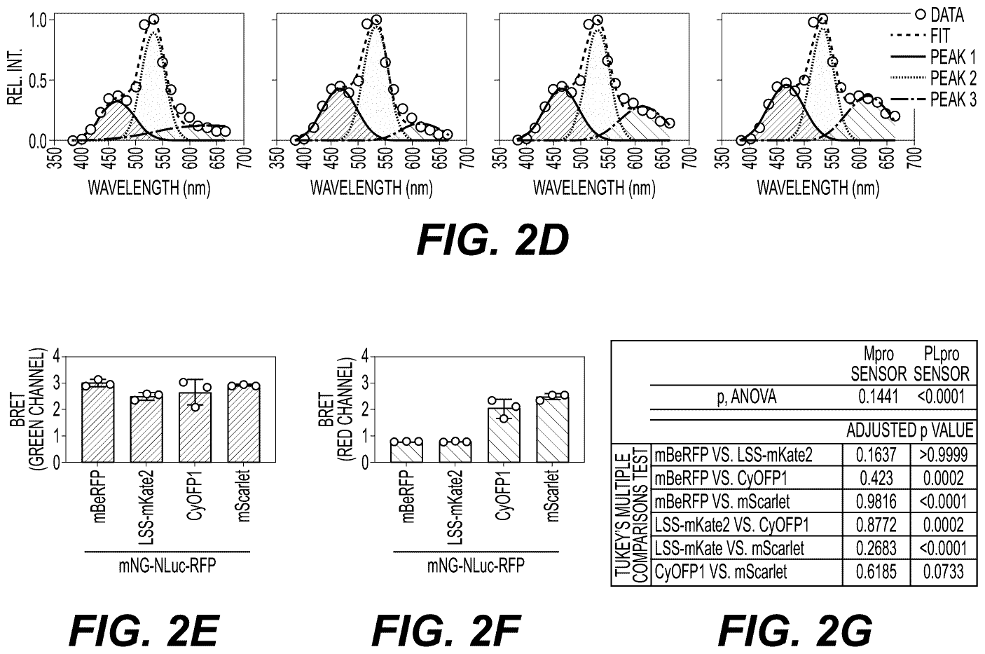

First, the long Stoke-shifted RFP, mBeRFP, has been reported to have a brightness of 17.6 but excellent excitation spectral overlap with NLuc emission spectra and largest separation of maximal excitation and emission wavelengths (λₑₓ and λₑₘ of 446 and 611 nm) (FIGS. 2A-2B). Second, the long Stoke-shifted RFP, LSS-mKate2, has a lesser brightness (4) but the good excitation spectral overlap with NLuc emission spectra as well as good separation of maximal excitation and emission wavelengths (λₑₓ and λₑₘ of 460 and 605 nm) (FIGS. 2A-2B). Third, the long stoke-shifted RFP, CyOFP1, has a relatively better brightness as well as excellent excitation spectral overlap with NLuc emission spectra (λₑₓ and λₑₘ of 497 and 589 nm) (FIGS. 2A-2B). Fourth, the bright RFP, mScarlet, has the highest fluorescence emission yield (brightness value of 70) but less excitation spectral overlap with NLuc emission spectra (λₑₓ and λₑₘ of 569 and 594 nm) (FIGS. 2A-2B).

TABLE 1
Spectral and photophysical properties of selected RFPs
used to generate various DuProSense constructs.

| | | | EC | | | |
|---|---|---|---|---|---|---|
| | | | (M⁻¹ | | | Matu- |
| | λex | λem | cm⁻¹) × | Quantum | Bright- | ration |
| | (nm) | (nm) | 10³ | Yield | ness | (min) |
| mBeRFP | 446 | 611 | 65 | 0.3 | 18 | 60 |
| LSS-mKate2 | 460 | 605 | 26 | 0.2 | 4 | 150 |
| CyOFP1 | 497 | 589 | 40 | 0.8 | 30 | 15 |
| mScarlet | 569 | 594 | 100 | 0.7 | 70 | 174 |

### Example 2. Spectral Characterization of Various RFP-Containing DuProSense Constructs

To spectrally characterize mBeRFP, LSS-mKate2, CyOFP1 and mScarlet containing DuProSense constructs, we transfected human embryonic kidney HEK293T cells with the sensor plasmid constructs, prepared cell lysates, and measured both fluorescence and bioluminescence spectra (FIGS. 2C-2D). Fluorescence spectra acquired using 440 nm excitation wavelength showed a peak at ˜533 nm, corresponding to mNG, for all constructs (FIG. 2C). Additionally, a second peak corresponding to the RFPs could also be observed (FIGS. 2B-2C) confirming the expression of correct dual sensor constructs. We note that in addition to the possibility of direct excitation of various RFPs, this may also indicate FRET-based energy transfer from mNG to the RFPs. Following this, we measured the bioluminescence spectra of each DuProSense construct after the addition of NLuc substrate. This revealed emission peaks corresponding to NLuc, mNG, and the RFP, although the amplitudes of RFP emission varied between the constructs and were much lower than that of mNG (FIG. 2B). Further, we determined BRET efficiencies of both the green channel biosensor as well as the red channel biosensors, after considering any FRET between mNG and the RFP (see e.g., Example 1) (FIGS. 2E-2G). This indicates that RET efficiencies for NLuc and mNG (green channel biosensor module) are much higher (3.00±0.12, 2.49±0.14, 2.65±0.50 and 2.92±0.03, respectively for mBeRFP, LSS-mKate2, CyOFP1 and mScarlet-containing DuProSense constructs; mean±s.d.; N=3) compared to those between NLuc and RFP (red channel biosensor module) (0.06±0.00, 0.06±0.00, 0.16±0.03 and 0.19±0.01, respectively for mBeRFP, LSS-mKate2, CyOFP1 and mSca-containing DuProSense constructs; N=3) in each of the DuProSense constructs. It was found that while green channel biosensor BRET efficiency remained constant, the red channel biosensor BRET efficiency was found to be high for the DuProSense constructs containing CyOFP1 and mScarlet as the RFP acceptor likely highlighting a role for quantum yield of the RFP (0.8 and 0.7 for CyOFP1 and mScarlet, respectively, as compared to 0.3 and 0.2 for mBeRFP and LSS-mKate2, respectively; Pearson r=0.93; Table 1) in determining BRET efficiency.

### Example 3. Simultaneous Detection of SARS-CoV-2 M^{pro} and PL^{pro}-Mediated Proteolytic Cleavage Using Various RFP-Containing DuProSense Constructs

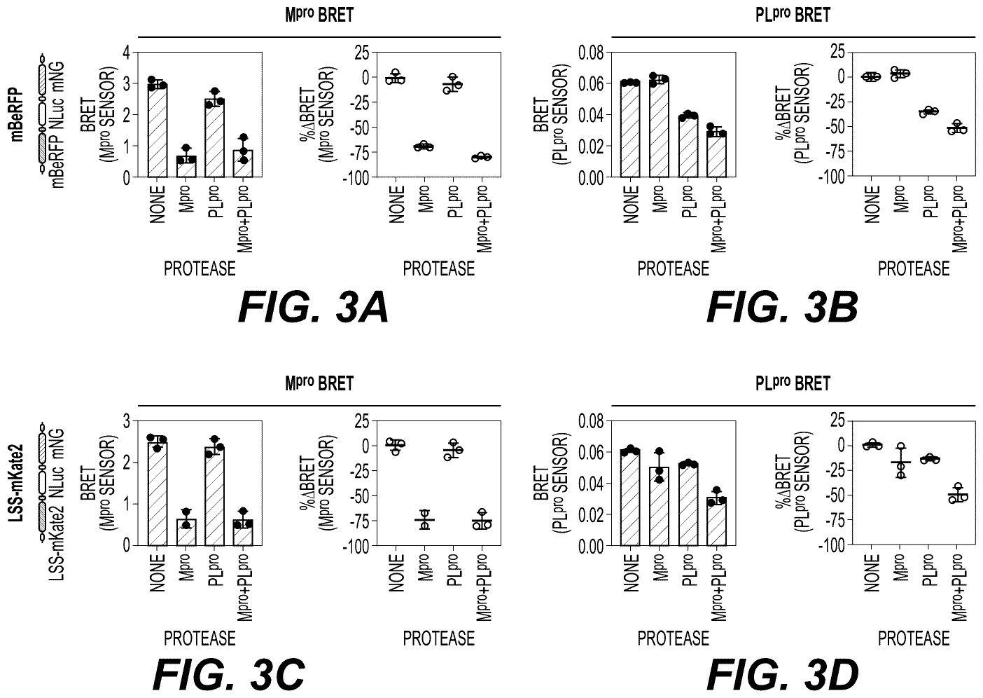

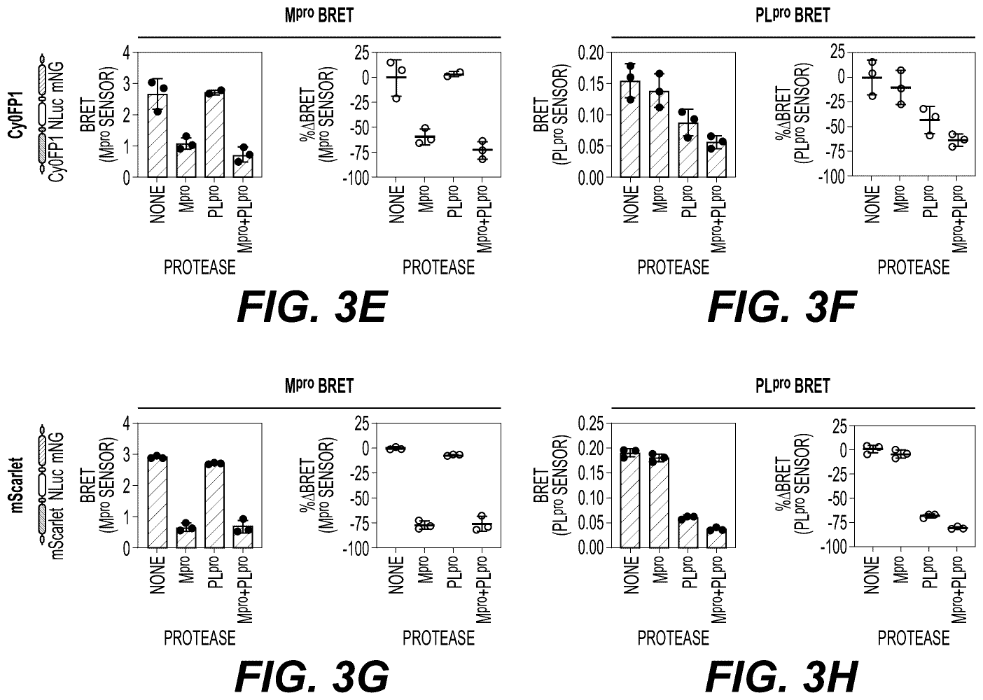

Following spectral characterization of various RFP-containing DuProSense biosensor constructs in vitro, we aimed to simultaneously detect the cleavage activity of two proteases in live cells. For this, we included the SARS-CoV-2 M^{pro} N-terminal autocleavage sequence (AVLQSGFR SEQ ID NO: 9) between mNG and NLuc (green channel biosensor) and PL^{pro} N-terminal autocleavage sequence (LKGGAPTK SEQ ID NO: 11) between NLuc and RFPs (red channel biosensor). We then transfected HEK293T cells with various RFP-containing DuProSense constructs along with either M^{pro} or PL^{pro} or both and monitored BRET in both the green (M^{pro} biosensor) as well as the red (PL^{pro}) channels. Expression of M^{pro} alone with the DuProSense constructs resulted in ˜75% decrease in BRET efficiency in the green channel (M^{pro} biosensor module) while expression of PL^{pro} alone did not result in such changes (FIGS. 3A, 3C, 3E, 3G) Expression of both M^{pro} and PL^{pro} resulted in similar decreases (˜75%) in the BRET efficiency in the green channel (FIGS. 3A, 3C, 3E, 3G). On the other hand, expression of M^{pro} alone did not result in decreases in BRET efficiency in the red channel (PL^{pro} biosensor module) as did expression of PL^{pro} alone while expression of both M^{pro} and PL^{pro} resulted in a large decrease in the BRET efficiency in the red channel (FIGS. 3B, 3D, 3F, 3H). The decrease in BRET efficiency in the red channel was found to vary, with the LSS-mKate2-containing DuProSense showing a minimal decrease, and the mScarlet-containing DuProSense showing a maximal decrease.

### Example 4. Western Blot Analysis of SARS-CoV-2 M^{pro} and PL^{pro}-Mediated Proteolytic Cleavage of Various RFP-Containing DuProSense Constructs

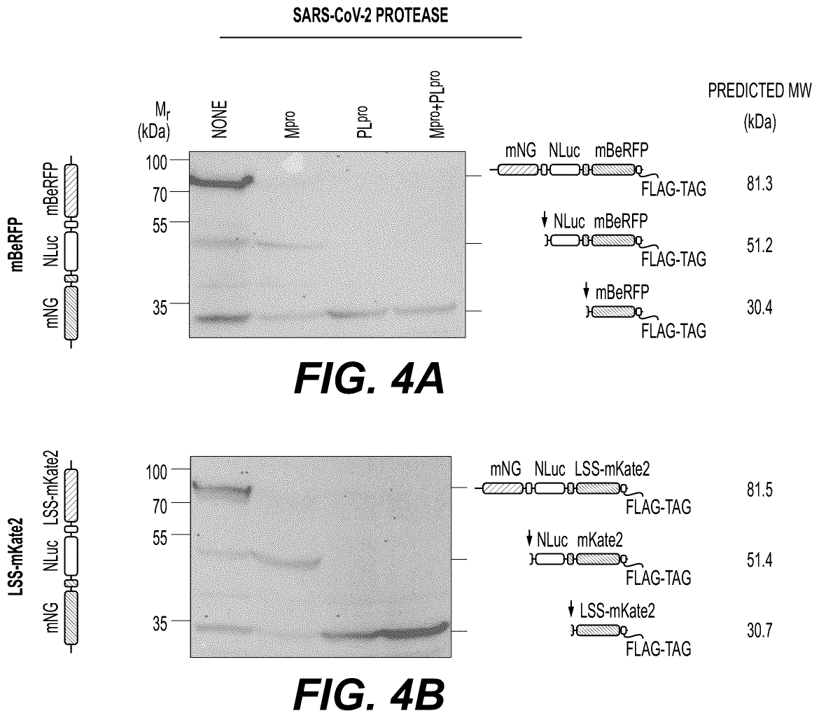

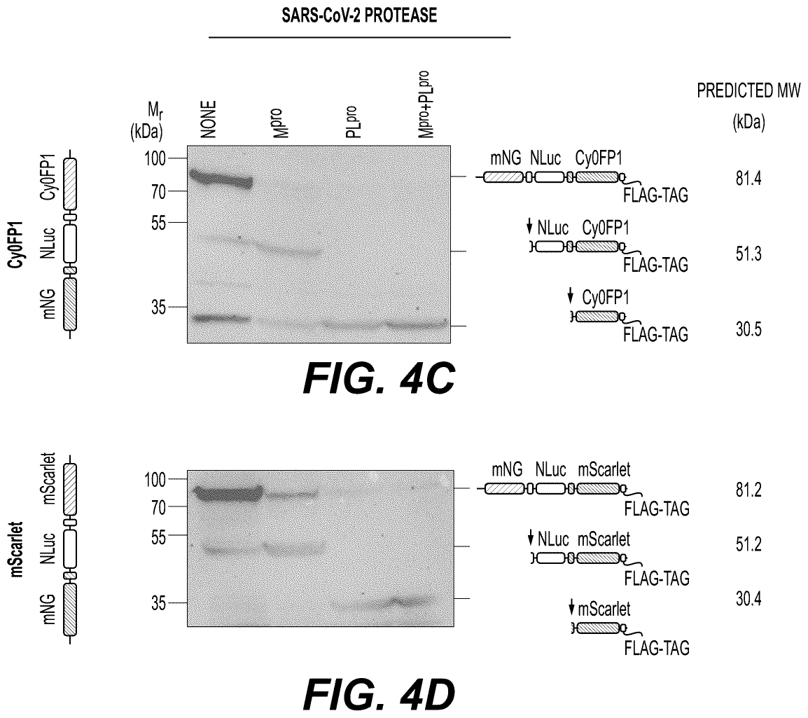

In order to confirm if the decreases in BRET efficiencies of the DuProSense constructs in the presence of either M^{pro} or PL^{pro} or both are due to cleavage of the proteins, we performed western blot analysis using an anti-FLAG-tag antibody. Each of the four DuProSense constructs contained a C-terminal FLAG-tag following RFP sequences and therefore, either M^{pro} or PL^{pro}-mediated cleavage of the biosensor constructs will result in the appearance of bands with smaller molecular weights. It was found that expression of M^{pro} in cells along with the four DuProSense constructs resulted in the loss of the ˜81 kDa band corresponding to the intact DuProSense construct with a concomitant appearance of a ˜51 kDa band corresponding to the NLuc-RFP fragment (FIGS. 4A-4D). Similarly, the expression of PL^{pro} resulted in the loss of the band corresponding to the intact DuProSense constructs with a concomitant appearance of a ˜30 kDa band corresponding to respective RFPs (FIGS. 4A-4D). Expression of both M^{pro} and PL^{pro} also resulted in a loss of the band corresponding to the intact DuProSense construct with the concomitant appearance of a ˜30 kDa band corresponding to respective RFPs. While these results unambiguously revealed a proteolytic cleavage of the four DuProSense constructs, we noticed significant non-specific cleavage of the mBeRFP, LSS-mKate2 and CyOFP1-containing DuProSense constructs in the absence of the two SARS-CoV-2 proteases as evidenced by the appearance of a ˜30 kDa band in the blots (FIGS. 4A-4C) while such a band could not be observed with the mScarlet-containing DuProSense construct. Thus, based on the data presented above i.e., BRET ratios in the green and red channels (high BRET in the red channel), percentage decrease in BRET efficiency in the presence of protease (maximal decrease in the red channel BRET efficiency in the presence of PL^{pro}) and western blot analysis (no non-specific cleavage), the further experiments utilized the mScarlet-containing DuProSense.

### Example 5. Mutation of SARS-CoV-2 M^{pro} and PL^{pro} Cleavage Sites Results in Abrogation of DuProSense BRET Response

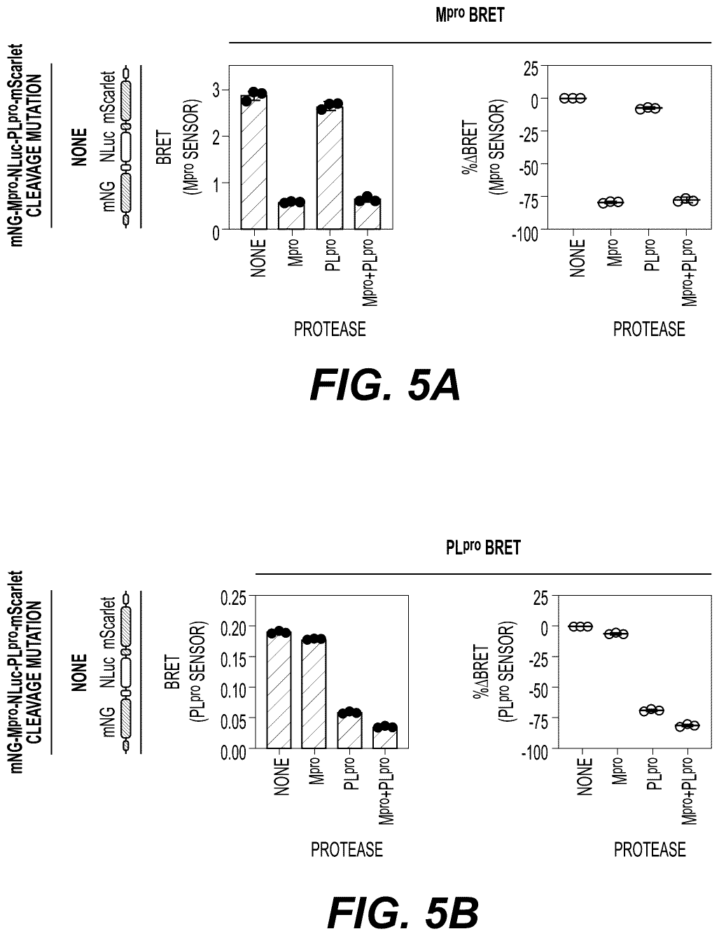

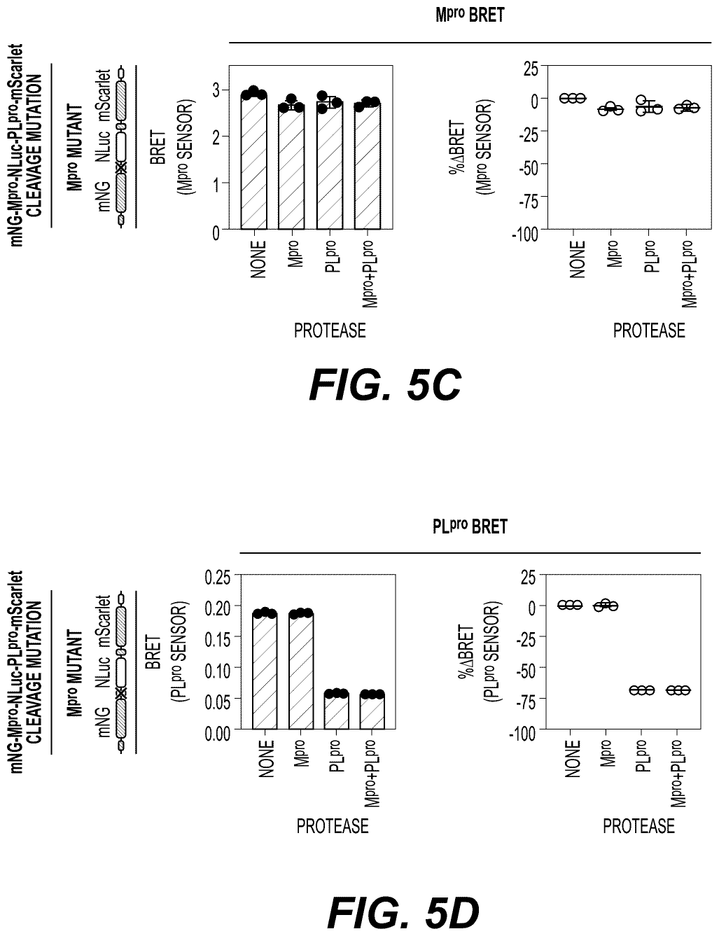

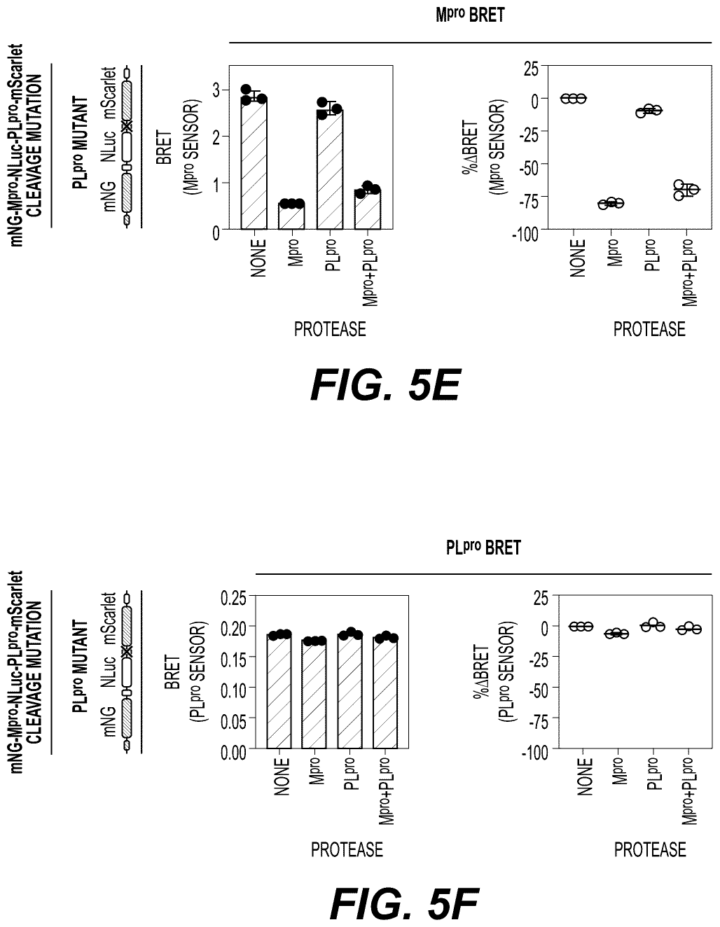

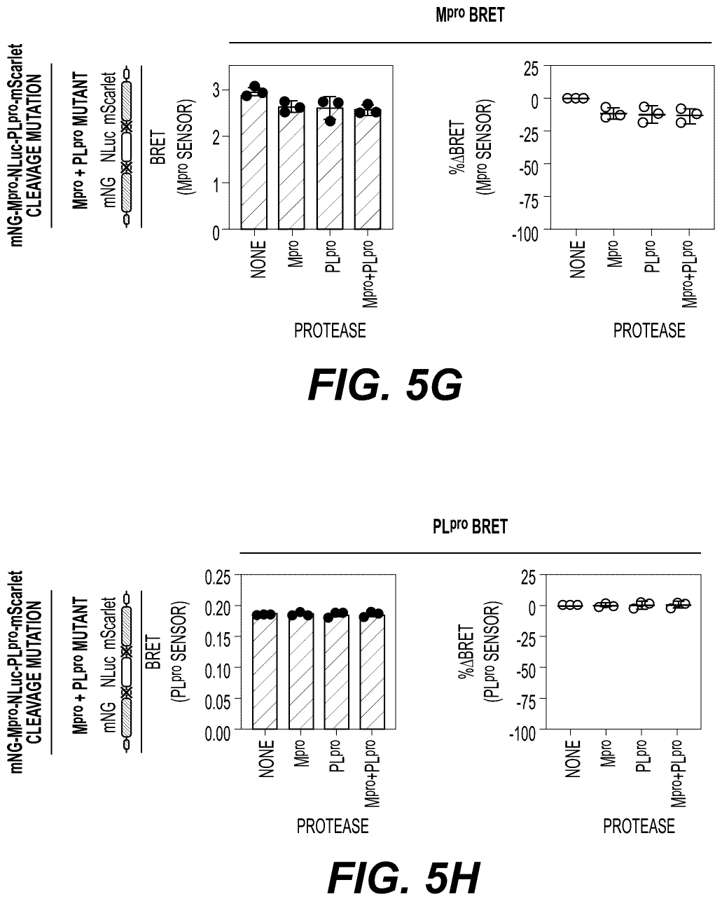

Using the mScarlet-based DuProSense, we determined the specificity of SARS-CoV-2 M^{pro} and PL^{pro}-mediated cleavage of the biosensor. For this, we generated mutations in either the M^{pro} cleavage site or the PL^{pro} cleavage site or both through the mutation of the critical Gln (fourth residue in the M^{pro} cleavage site) to Ala (from AVLQSGFR SEQ ID NO: 9 to AVLASGFR SEQ ID NO: 13) and Gly (fourth residue in the PL^{pro} cleavage site) to lie (from LKGGAPTK SEQ ID NO: 11 to LKGIAPTK SEQ ID NO: 15). We then expressed the wild type and mutant mScarlet-based DuProSense biosensor along with either M^{pro} alone or PL^{pro} alone or both proteases. While expression of M^{pro} resulted in a large decrease in the green channel (M^{pro} biosensor module) BRET efficiency of the wild type as well as the PL^{pro} cleavage site mutant DuProSense biosensor, such decreases were not seen with M^{pro} cleavage site mutant (M^{pro} as well as both M^{pro} and PL^{pro} cleavage site mutant DuProSense biosensor) (FIGS. 5A, 5C, 5E, 5G). Similarly, while expression of PL^{pro} resulted in a large decrease in the red channel (PL^{pro} biosensor module) BRET efficiency of the wild type as well as the M^{pro} cleavage site mutant DuProSense biosensor, such decreases were not seen with the PL^{pro} cleavage site mutant (PL^{pro} as well as both M^{pro} and PL^{pro} cleavage site mutant DuProSense biosensor) (FIGS. 5B, 5D, 5F, 5H). Expression of both M^{pro} and PL^{pro} in the cells specifically resulted in a decrease in BRET efficiency of wild-type and PL^{pro} cleavage site mutant DuProSense biosensor in the green channel (M^{pro} biosensor module) and wild-type and M^{pro} cleavage site mutant DuProSense biosensor in the red channel (PL^{pro} biosensor module). On the other hand, mutation of either the M^{pro} or PL^{pro} cleavage resulted in a loss in the BRET efficiency decrease M^{pro} or PL^{pro} or both M^{pro} and PL^{pro} cleavage site mutants (FIG. 5). Overall, these results indicate that the response of DuProSense biosensor in the form of a decrease in BRET efficiency in the presence of either M^{pro} or PL^{pro} or both proteases is highly specific.

### Example 6. Monitoring Pharmacological Inhibition of SARS-CoV-2 M^{pro} and PL^{pro} Proteases Using DuProSense

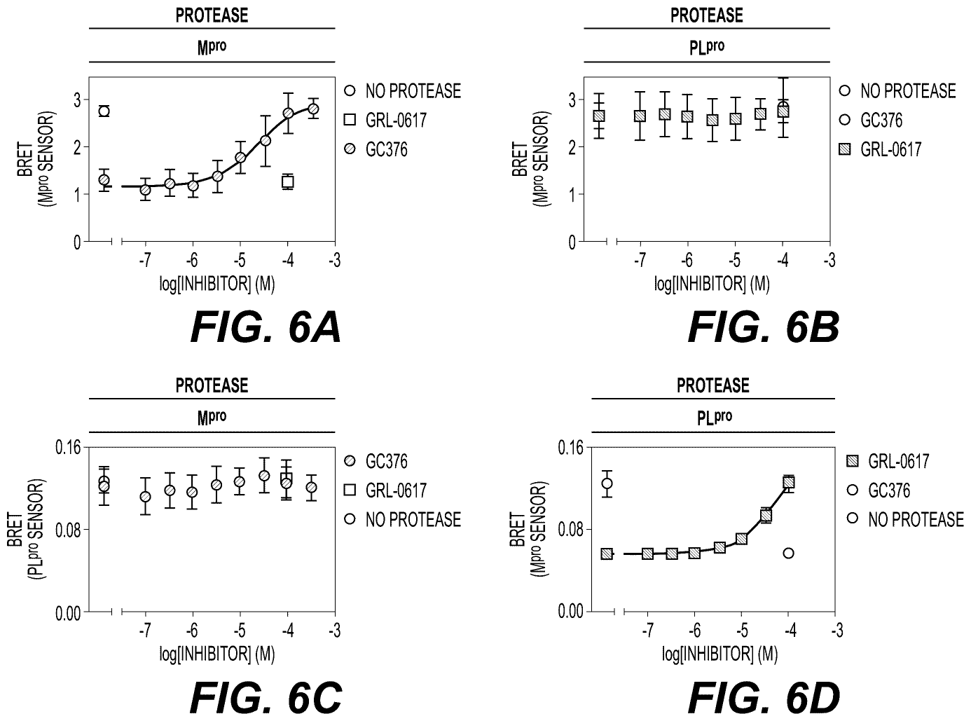

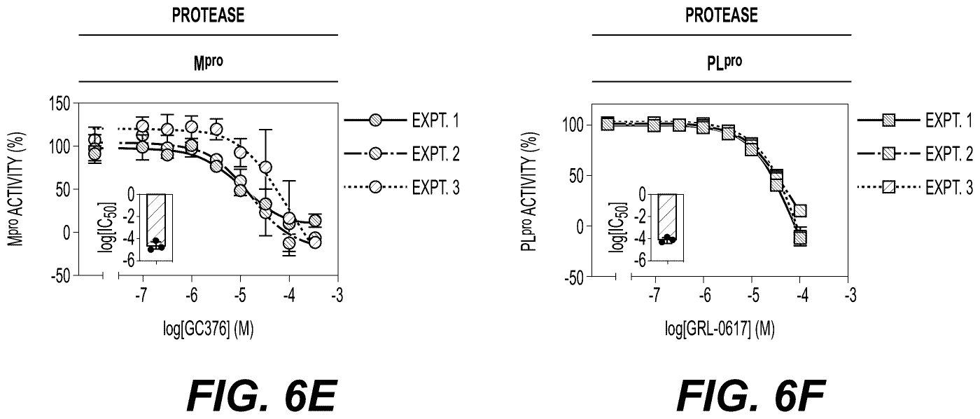

Having established the specificity of proteolytic cleavage of the DuProSense biosensor, we then monitored the inhibition of SARS-CoV-2 M^{pro} and PL^{pro} proteases in living cells. For this, we transfected cells with the mScarlet-containing DuProSense biosensor along with either M^{pro} or PL^{pro} proteases and treated the cells with a range of concentrations of either GC376 (M^{pro} inhibitor) or GRL-0617 (PL^{pro} inhibitor). We then measured BRET efficiency of the DuProSense biosensor in both the green channel (M^{pro} biosensor module) as well as the red channel (PL^{pro} biosensor module). This revealed a GC376 concentration-dependent increase in the green channel BRET efficiency in cells expressing M^{pro} protease, indicative of an inhibition of the M^{pro} protease, (FIG. 6A) whereas red channel BRET efficiency in the same cells remained high (FIG. 6C). Similarly, while the green channel BRET remained high (FIG. 6B), a GRL-0617 concentration-dependent increase in the red channel BRET efficiency in cells expressing PL^{pro} protease, indicative of an inhibition of PL^{pro} protease in the cells (FIG. 6D). Further, this revealed an IC₅₀ value of 13.9±2.9 μM of GC376 against M^{pro} and 77.2±34.8 μM of GRL-0617 against PL^{pro} (FIGS. 6E-6F). Thus, the high specificity of signal changes observed with DuProSense allowed us to unambiguously monitor the pharmacological inhibition of the two SARS-CoV-2 proteases, M^{pro} and PL^{pro}.

We have engineered a genetically encoded, BRET-based, two-color, dual protease biosensor, DuProSense, that can be used for simultaneous monitoring of the proteolytic activity of two proteases in living cells. We have extensively characterized the design using SARS-CoV-2 M^{pro} and PL^{pro} in terms of specificity of signal upon protease-mediated cleavage as well as pharmacological inhibition of the proteases. DuProSense can be directly utilized in including (i) drug discovery projects directed towards the development of either SARS-CoV-2 M^{pro} and PL^{pro} individual or dual-protease (bispecific) inhibitors, (ii) functional analysis of SARS-CoV-2 genomic variants in the M^{pro} and PL^{pro} coding regions and (iii) monitoring the impact of human host factor interaction with M^{pro} and PL^{pro}. Thus, the biosensor will be useful in fighting against the current as well as future threats from SARS-CoV-2 (and similar beta-coronaviruses) and their ever-evolving variants. More importantly, given the modular design, DuProSense can be extended in a straightforward manner to any pair of proteases, including human host proteases, enabling simultaneous monitoring of more than one signaling pathways in diseases such as pathogenic infections or cancer.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventors expect skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic characteristic(s). Embodiments of the invention so claimed are inherently or expressly described and enabled herein.

Furthermore, numerous references have been made to patents and printed publications throughout this specification and/or Exhibit A. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A bioluminescence resonance energy transfer (BRET) sensor comprising a single polypeptide chain comprising:
1) a bioluminescent protein, or a mutant thereof;
2) a first BRET acceptor sequence comprising a monomeric NeonGreen (mNG) fluorescent protein; and
3) a second BRET acceptor sequence comprising a red fluorescent protein (RFP);
wherein the sensor detects activity of two proteins and wherein a first protein cleaves the first acceptor producing a change in green emission and the second protein cleaves the second acceptor producing a change in red emission.

2. The sensor of claim 1, wherein the bioluminescent protein is a luciferase, or an aequorin.

3. The sensor of claim 1, wherein the bioluminescent protein is selected from Renilla luciferase, firefly luciferase, and Gaussia luciferase, or a mutant thereof.

4. The sensor of claim 1, wherein the bioluminescent protein is selected from a nano-luciferase (NLuc), or a pic-luciferase (picALuc).

5. The sensor of claim 1, wherein the mNG fluorescent protein is attached to the N-terminal of the bioluminescent protein.

6. The sensor of claim 1, wherein the RFP is attached to the C-terminal of the bioluminescent protein.

7. The sensor of claim 1, wherein the RFP is selected from CyOFP1, mScarlet, LSS-mKate2, or mBeRFP tagged protein.

8. The sensor of claim 1, wherein:
(i) the bioluminescent protein has an emission spectrum with a peak emission;
(ii) the mNG fluorescent protein has a first excitation spectrum that overlaps with the emission spectrum; and
(iii) the RFP has a second excitation spectrum that overlaps with the emission spectrum, wherein the first excitation spectrum is separated from the second excitation spectrum.

9. The sensor of claim 8, wherein the second excitation spectrum is separated from the first excitation spectrum by about 10 nm to about 100 nm.

10. The sensor of claim 8, wherein the second excitation spectrum is separated from the first excitation spectrum by about 10 nm to about 50 nm.

11. The sensor of claim 8, wherein the second excitation spectrum is separated from the first excitation spectrum by about 10 nm to about 30 nm.

12. The sensor of claim 8, wherein the second excitation spectrum is separated from the first excitation spectrum by about 10 nm.

13. The sensor of claim 1, wherein the sensor detects proteolytic cleavage by two proteases.

14. The sensor of claim 13, wherein the sensor detects proteolytic cleavage of a coronavirus main protease (M^{pro}) and a coronavirus papain-like protease (PL^{pro}).

15. The sensor of claim 1, wherein the mNG fluorescent protein is a mNG coronavirus M^{pro} protease, and the RFP is an RFP coronavirus PL^{pro} protease.

16. The sensor of either one of claim 14 or 15, wherein the coronavirus is SARS-CoV-2.

17. A method for detecting coronavirus infection in a patient, the method comprising
(i) contacting a biological sample from the patient with the sensor of claim 15;
(ii) adding a substrate for the bioluminescent protein to the mixture; and
(iii) measuring fluorescence in a green channel and a red channel;
wherein
a reduction in the green channel fluorescence of the sensor indicates the presence of the coronavirus M^{pro} protease;
a reduction in the red channel fluorescence of the sensor indicates the presence of the coronavirus PL^{pro} protease; and
wherein the detection of coronavirus M^{pro} protease or coronavirus PL^{pro} protease, or both, indicates that the patient has SARS-CoV-2 infection.

18. A method for simultaneously testing inhibitors of coronavirus main protease (M^{pro}) and coronavirus papain-like protease (PL^{pro}), the method comprising
(i) contacting a test compound with a mixture comprising a coronavirus M^{pro} and a coronavirus PL^{pro};
(ii) adding a dual protease sensor of claim 15 to the mixture;
(iii) adding a substrate for the bioluminescent protein to the mixture; and
(iii) measuring fluorescence in a green channel and a red channel.

19. The method of claim 18, wherein the coronavirus is SARS-CoV-2.

20. The method of claim 18, wherein activity of a coronavirus M^{pro} protease results in the cleavage of the sensor resulting in a decrease in the BRET between the bioluminescent protein and the mNG protein, thereby reducing the green fluorescence of the sensor, and indicating the presence of the coronavirus M^{pro} protease; and
wherein if the compound inhibits the coronavirus M^{pro} protease, the green fluorescence of the sensor will not be reduced.

21. The method of claim 18, wherein activity of a coronavirus PL^{pro} protease results in the cleavage of the sensor resulting in a decrease of BRET between the bioluminescent protein and the RFP, thereby reducing the red fluorescence of the sensor, and indicating the presence of the coronavirus PL^{pro} protease; and
wherein if the compound inhibits the coronavirus PL^{pro} protease, the red fluorescence of the sensor will not be reduced.

22. A dual protease sensor for detecting a coronavirus comprising an amino acid construct of SEQ ID NO: 1, SEQ ID NO: 3, SEQ ID NO: 5, or SEQ ID NO: 6.

23. A plasmid comprising a nucleotide construct of SEQ ID NO: 2, SEQ ID NO: 4, SEQ ID NO: 6, or SEQ ID NO: 8.

24. A cell transfected with a plasmid of claim 20.

25. A kit comprising the sensor of claim 1, and a substrate for the bioluminescent protein.
